(19) United States
(12) Patent Application Publication
(10) Pub. No.: US 2016/0258959 A1
(43) Pub. Date: Sep. 8, 2016

(54) METHODS OF DETERMINING A PATIENT'S PROGNOSIS FOR RECURRENCE OF PROSTATE CANCER AND/OR DETERMINING A COURSE OF TREATMENT FOR PROSTATE CANCER FOLLOWING A RADICAL PROSTATECTOMY

(71) Applicant: Quanterix Corporation, Lexington, MA (US)

(72) Inventors: David Wilson, Boxborough, MA (US); David C. Duffy, Arlington, MA (US); David Hanlon, Derry, NH (US); David Okrongly, Fort Lauderdale, FL (US)

(73) Assignee: Quanterix Corporation, Lexington, MA (US)

(21) Appl. No.: 14/963,421

(22) Filed: Dec. 9, 2015

Related U.S. Application Data
(63) Continuation of application No. 14/124,807, filed on Apr. 23, 2014, now abandoned.
(60) Provisional application No. 61/495,355, filed on Jun. 9, 2011.

(51) Int. Cl. G01N 33/574

(52) U.S. Cl. CPC G01N 33/57434; G01N 2800/56; G01N 2800/52; G01N 2333/96455

(57) ABSTRACT

The present invention generally relates, in some embodiments, to methods of determining a patient's prognosis for recurrence of prostate cancer and/or determining a course of treatment for prostate cancer following a radical prostatectomy.

### RELATED APPLICATIONS

This application is a continuation of U.S. patent application Ser. No. 14/124,807, filed Apr. 23, 2014, and entitled “METHODS OF DETERMINING A PATIENT′S PROGNOSIS FOR RECURRENCE OF PROSTATE CANCER AND/OR DETERMINING A COURSE OF TREATMENT FOR PROSTATE CANCER FOLLOWING A RADICAL PROSTATECTOMY,” which is a national stage of International Patent Application Serial No. PCT/US2012/041489, filed Jun. 8, 2012, and entitled “METHODS OF DETERMINING A PATIENT′S PROGNOSIS FOR RECURRENCE OF PROSTATE CANCER AND/OR DETERMINING A COURSE OF TREATMENT FOR PROSTATE CANCER FOLLOWING A RADICAL PROSTATECTOMY,” which claims priority under 35 U.S.C. §119(e) to U.S. Provisional Patent Application Ser. No. 61/495,355, filed Jun. 9, 2011, and entitled “METHODS OF DETERMINING A PATIENT′S PROGNOSIS FOR RECURRENCE OF PROSTATE CANCER AND/OR DETERMINING A COURSE OF TREATMENT FOR PROSTATE CANCER FOLLOWING A RADICAL PROSTATECTOMY,” each of which is incorporated herein by reference.

### FIELD

Disclosed are methods of determining a patient's prognosis for recurrence of prostate cancer and/or determining a course of treatment for prostate cancer, following a radical prostatectomy.

### BACKGROUND

Prostate cancer is one of the most common types of cancer in men. A common treatment for men with prostate cancer is a radical prostatectomy which is an operation to remove the prostate gland and some of the tissue around it. A radical prostatectomy removes the tissue responsible for the majority of prostate specific antigen (PSA) production and thus, post-surgical PSA in a patient is usually present at very low levels in the months following a radical prostatectomy. In some patients, following a radical prostatectomy, PSA levels rise with time (e.g., over months to years) which can indicate a return of the patient's prostate cancer. While methods and systems exist for detecting the recurrence of prostate cancer using PSA levels, most current assay methods are unable to detect low quantities of PSA and thus PSA is not detected in a patient sample(s) until a significant period of time has elapsed following a radical prostatectomy (e.g., until PSA levels rise to a level which is detectable by the assay). Therefore, early detection of recurrence of prostate cancer is not available based on a PSA level. In addition, many assays do not have the precision and/or limits of detection needed to allow for a low PSA level to be indicative of recurrence and thus, multiple samples must be collected from a patient over time and analyzed together (e.g., by determining an increase in PSA levels over time) to be indicative of prostate cancer recurrence. Accordingly, improved methods and systems are needed

### SUMMARY

In some embodiments, a method of determining a patient's prognosis for recurrence of prostate cancer and/or determining a course of treatment for prostate cancer, following a radical prostatectomy comprises performing an assay on a sample obtained from the patient following the radical pro statectomy to determine a measure of the concentration of prostate specific antigen (PSA) in the sample, wherein the concentration of PSA in the sample is less than about 50 pg/mL; and determining the patient's prognosis for recurrence of prostate cancer and/or determining a course of treatment for prostate cancer following the radical prostatectomy based at least in part on the measured concentration of PSA in the sample, wherein determining the patient's prognosis for recurrence of prostate cancer and/or determining a course of treatment does not require measurement of a change in concentration of PSA measured in multiple patient samples as a function of time elapsed after the radical prostatectomy.

In some embodiments, a method of determining a patient's prognosis for recurrence of prostate cancer and/or determining a course of treatment for prostate cancer, following a radical prostatectomy comprises determining a patient's prognosis for recurrence of prostate cancer and/or determining a course of treatment for prostate cancer following the radical prostatectomy based at least in part on a concentration of

PSA measured in a sample by an assay performed on the sample obtained from the patient following the radical prostatectomy to determine the measure of the concentration of PSA in the sample, wherein the concentration of PSA in the sample is less than about 50 pg/mL, and wherein determining the patient's prognosis for recurrence of prostate cancer and/or determining a course of treatment does not require measurement of a change in concentration of PSA measured in multiple patient samples as a function of time elapsed after the radical prostatectomy.

In some embodiments, a method for performing an assay and providing data for determining a patient's prognosis for recurrence of prostate cancer and/or determining a course of treatment for prostate cancer following a radical prostatectomy comprises performing an assay on a sample obtained from the patient following the radical prostatectomy to determine a measure of the concentration of PSA in the sample, wherein the concentration of PSA in the sample is less than about 50 pg/mL; and providing data from the assay to enable determining the patient's prognosis for recurrence of prostate cancer and/or determining a course of treatment for prostate cancer, following the radical prostatectomy, based at least in part on the measured concentration of PSA in the sample, wherein the data does not include measurement of a change in concentration of PSA measured in multiple patient samples as a function of time elapsed after the radical prostatectomy.

In some embodiments, a method of determining a patient's prognosis for recurrence of prostate cancer, and/or determining a course of treatment for prostate cancer following a radical prostatectomy comprises performing an assay on a sample obtained from the patient following the radical pro statectomy to determine a measure of the concentration of prostate specific antigen (PSA) in the sample, wherein the sample is obtained from the patient within 6 months following the radical prostatectomy; and determining the patient's prognosis for recurrence of prostate cancer and/or determining a course of treatment for prostate cancer following a radical prostatectomy based at least in part on the concentration of PSA measured in the sample, wherein determining the patient's prognosis for recurrence of prostate cancer and/or determining a course of treatment does not require measurement of a change in concentration of PSA measured in multiple patient samples as a function of time elapsed after the radical prostatectomy.

In some embodiments, a method of determining a patient's prognosis for recurrence of prostate cancer and/or determining a course of treatment for prostate cancer, following a radical prostatectomy comprises determining a patient's prognosis for recurrence of prostate cancer and/or determining a course of treatment for prostate cancer following the radical prostatectomy based at least in part on a concentration of PSA measured in a sample by an assay performed on the sample obtained from the patient following the radical prostatectomy to determine the measure of the concentration of PSA in the sample, wherein the sample is obtained from the patient within 6 months following the radical prostatectomy, and wherein determining the patient's prognosis for recurrence of prostate cancer and/or determining a course of treatment does not require measurement of a change in concentration of PSA measured in multiple patient samples as a function of time elapsed after the radical prostatectomy.

In some embodiments, a method for performing an assay and providing data for determining patient's prognosis for recurrence of prostate cancer, and/or determining a course of treatment for prostate cancer following a radical prostatectomy comprises performing an assay on a sample obtained from the patient following the radical prostatectomy to determine a measure of the concentration of PSA in the sample, wherein the sample is obtained from the patient within 6 months following the radical prostatectomy; and providing data from the assay to enable determining the patient's prognosis for recurrence of prostate cancer and/or determining a course of treatment for prostate cancer, following a radical prostatectomy, based at least in part on the concentration of PSA measured in the sample, wherein determining the patient's prognosis for recurrence of prostate cancer and/or determining a course of treatment does not require measurement of a change in concentration of PSA measured in multiple patient samples as a function of time elapsed after the radical prostatectomy.

In some embodiments, a method of determining a patient's prognosis for recurrence of prostate cancer, and/or determining a course of treatment for prostate cancer following a radical prostatectomy comprises performing an assay on at least one sample obtained from the patient following the radical prostatectomy to determine a measure of the concentration of prostate specific antigen (PSA) in the at least one sample; and determining the patient's prognosis for recurrence of prostate cancer and/or determining a course of treatment for prostate cancer following a radical prostatectomy based at least in part on the concentration of PSA measured in the at least one sample, wherein a measured concentration of PSA in the at least one sample greater than a threshold limit of no greater than about 10 pg/mL indicates a significant likelihood that the patient's prostate cancer will reoccur within 5 years. In some embodiments, a method of determining a patient's prognosis for recurrence of prostate cancer and/or determining a course of treatment for prostate cancer, following a radical prostatectomy comprises determining a patient's prognosis for recurrence of prostate cancer and/or determining a course of treatment for prostate cancer, following a radical prostatectomy based at least in part on a concentration of PSA measured in at least one sample by an assay performed on the at least one sample obtained from the patient following the radical pro statectomy to determine the measure of the concentration of PSA in the at least one sample, wherein a measured concentration of PSA greater than about 10 pg/mL in the at least one sample indicates a significant likelihood that the patient's prostate cancer will reoccur within 5 years.

In some embodiments, a method for performing an assay and providing data for determining patient's prognosis for recurrence of prostate cancer, and/or determining a course of treatment for prostate cancer following a radical prostatectomy comprises performing an assay on at least one sample obtained from the patient following the radical prostatectomy to determine a measure of the concentration of PSA in the at least one sample; and providing data from the assay to enable determining the patient's prognosis for recurrence of prostate cancer and/or determining a course of treatment for prostate cancer, following a radical prostatectomy, based at least in part on the concentration of PSA measured in the at least one sample, wherein a measured concentration of PSA greater than a threshold limit of no greater than about 10 pg/mL indicates a significant likelihood that the patient's prostate cancer will reoccur within 5 years.

In some embodiments, a method of determining a patient's prognosis for recurrence of prostate cancer, and/or determining a course of treatment for prostate cancer following a radical prostatectomy comprises performing an assay on at least one sample obtained from the patient following the radical prostatectomy to determine a measure of the concentration of prostate specific antigen (PSA) in the at least one sample; and determining the patient's prognosis for recurrence of prostate cancer and/or determining a course of treatment for prostate cancer following a radical prostatectomy based at least in part on the concentration of PSA measured in the at least one sample, wherein a measured concentration of PSA in the at least one sample less than a threshold limit of no greater than about 10 pg/mL indicates a significant likelihood that the patient's prostate cancer will not reoccur within 5 years.

In some embodiments, a method of determining a patient's prognosis for recurrence of prostate cancer and/or determining a course of treatment for prostate cancer, following a radical prostatectomy comprises determining a patient's prognosis for recurrence of prostate cancer and/or determining a course of treatment for prostate cancer, following a radical prostatectomy based at least in part on a concentration of PSA measured in at least one sample by an assay performed on the at least one sample obtained from the patient following the radical prostatectomy to determine the measure of the concentration of PSA in the at least one sample, wherein a measured concentration of PSA less than about 10 pg/mL in the at least one sample indicates a significant likelihood that the patient's prostate cancer will not reoccur within 5 years. In some embodiments, a method for performing an assay and providing data for determining patient's prognosis for recurrence of prostate cancer, and/or determining a course of treatment for prostate cancer following a radical prostatectomy comprises performing an assay on at least one sample obtained from the patient following the radical prostatectomy to determine a measure of the concentration of PSA in the at least one sample; and providing data from the assay to enable determining the patient's prognosis for recurrence of prostate cancer and/or determining a course of treatment for prostate cancer, following a radical prostatectomy, based at least in part on the concentration of PSA measured in the at least one sample, wherein a measured concentration of PSA less than a threshold limit of no greater than about 10 pg/mL indicates a significant likelihood that the patient's prostate cancer will not reoccur within 5 years.

### BRIEF DESCRIPTION OF THE DRAWINGS

FIG. 1a is a schematic flow diagram depicting one embodiment of steps (A-D) for performing an exemplary method of the present invention; and

FIG. 1b is a schematic flow diagram depicting one embodiment of steps (A-D) for performing an exemplary method of the present invention.

FIG. 2 shows a graph of the average enzymes per bead versus concentration of PSA, according to an assay performed using an exemplary method of the present invention;

FIG. 3a highlights the low background obtained with digital quantification, according to some embodiments;

FIG. 3b depicts the linearity obtained from admixtures of high and low female serum samples, accordingly to some embodiments; FIG. 4 shows a plot of the %CV versus PSA concentration for a plurality of samples measured using an exemplary assay method of the present invention;

FIG. 5 shows a plot of the PSA concentration measured in a plurality of samples on a plurality of days using an exemplary assay method;

FIG. 6 shows a plot comparing PSA concentrations in a plurality of samples measured using two assay methods;

FIG. 7 shows a plot of the PSA concentrations measured for radical prostatectomy (RP) patients with recurring and non-recurring prostate cancer;

FIG. 8a depicts PSA concentrations from non-recurring patients, according to some embodiments of the present invention;

FIG. 8b shows an expanded plot of a subset of patients from FIG. 8a.

FIG. 9 shows a plot by non-recurrence and recurrence groups of men following radical prostatectomy, according to an exemplary method of the present invention; and

FIG. 10 shows Kaplan Meier time to biochemical recurrence curves, accordingly to an exemplary method of the present invention.

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents incorporated herein by reference are incorporated by reference in their entirety. In case of conflict, the present specification, including definitions, will control.

### DETAILED DESCRIPTION

Described are inventive methods of determining a patient's prognosis for recurrence of prostate cancer and/or determining a course of treatment for prostate cancer following a radical prostatectomy. In some embodiments, the methods comprise determining a measure of the concentration of prostate specific antigen (PSA) in a patient sample containing or suspected of containing PSA. The subject matter of the present invention involves, in some cases, interrelated products, alternative solutions to a particular problem, and/or a plurality of different uses of one or more systems and/or articles.

In some embodiments, a method of the present invention comprises determining a measure of the concentration of PSA in at least one sample obtained from a patient following a radical prostatectomy. A prognostic indication of the patient's likelihood of recurrence of prostate cancer and/or determination of a course of treatment may be based at least in part on the measure of the concentration of the PSA present in the at least one sample. In some embodiments, the methods of the present invention make use of assay methods having very low limits of detection (“LODs”) and/or limits of quantification (“LOQs”) (e.g., in the low pg/mL range or less) to determine a measure of the concentration of a PSA in at least one sample obtained from a patient following a radical prostatectomy.

As known in the art, a radical prostatectomy is an operation which removes the prostate gland and some of the tissue around it and is a commonly used treatment for patients diagnosed with prostate cancer. Radical prostatectomy removes the tissue responsible for prostate specific antigen (PSA) production and thus, levels of PSA are generally low and/or undetectable following a radical prostatectomy. In some patients, following a radical prostatectomy, PSA levels increase with time (e.g., over months to years) which can indicate a return of the patient's prostate cancer.

As noted above, the present invention, in some embodiments, employs assay methods which have very low limits of quantification and/or limits of detection, and allow for the measurement of the concentration of PSA in patient samples. The ability to accurately and/or reproducibly measure extremely low levels of PSA in patient samples can allow for correlations to be made between PSA levels and the likely recurrence of prostate cancer for the patient and/or suitable courses of treatment (e.g., due to the likely possibility of recurrence of prostate cancer). Currently, it is not generally accepted that determining a low concentration (e.g., less than 100 pg/mL, less than 50 pg/mL, less than 20 pg/mL, less than 10 pg/mL, less than 5 pg/mL, etc.) of PSA in a patient sample would be useful to determine the likelihood of recurrence of prostate cancer because, for example, it is conventionally believed that such low levels may not distinguish between background PSA levels/noise. In addition, low levels of PSA may be present in the patient sample due to sources other than the prostate, for example, the periurethral glands, the perirectals glands, peripheral blood cells, and/or other peripheral tissues. In addition, there is controversy as to whether the concentration of PSA in at least one sample obtained from a patient at about a single time point (e.g., as opposed to using the change in concentration of PSA in a plurality of samples obtained from a patient over time) could be indicative of a recurrence of prostate cancer because, for example, such an analysis may not take into account background noise and/or baseline values for a patient. Accordingly, currently accepted clinical practice and guidelines focus on looking at levels of PSA generally exceeding about 100 pg/ml and/or at a rise or increase in PSA levels of a patient over time.

In some embodiments, the present invention provides methods for determining the likelihood that a patient's prostate cancer will reoccur at an earlier time point followed a radical prostatectomy as compared to current methods. Generally, cancer treatments are most effective if provided to a patient as soon as a cancer is detected, and thus, the ability to detect earlier a strong likelihood of recurrence is beneficial because treatment can be provided at an earlier time point, which can decrease the likelihood of the cancer spreading and/or the necessity of harsh treatment protocols. In some embodiments, the ability to determine whether a patient's prostate cancer is likely or unlikely to reoccur soon after a radical prostatetectomy may be used to determine whether the patient should receive additional treatment and/or whether such treatment is unnecessary. For example, if a patient's prostate cancer is determined to likely reoccur, the patient may undergo a treatment protocol in addition to the radical pro statectomy, such as radiation treatment, soon after the surgery.

In some embodiments, the present invention provides methods of determining a patient's prognosis for recurrence of prostate cancer and/or determining a course of treatment for prostate cancer, following a radical prostatectomy, based at least in part on the measured concentration of PSA from a sample obtained from the patient following the radical prostatectomy. In some cases, the method comprises performing an assay on a sample obtained from the patient following the radical prostatectomy to determine a measure of the concentration of PSA in the sample, and determining the patient's prognosis for recurrence of prostate cancer and/or determining a course of treatment for prostate cancer following the radical prostatectomy based at least in part on the measured concentration of PSA in the sample. Assay methods and systems capable of determining the concentration of PSA are described herein. In some cases, the measure concentration of PSA is less than about 50 pg/mL, or less than about 40 pg/mL, or less than about 30 pg/mL, or less than about 20 pg/mL, or less than about 10 pg/mL, or less than about 5 pg/mL, or less than about 3 pg/mL, or another suitable range or level as described herein.

In some embodiments, the measured concentration of PSA is the patient's nadir PSA. The term “nadir PSA” is given its ordinary meaning in the art and refers to the lowest PSA concentration obtained for a patient after a treatment for prostate cancer, including radical prostatectomy. Nadir values would be expected to differ for each patient and by type of treatment received (surgery, radiation etc). For prostatectomy, this means any elevation due to surgery should be allowed to clear from circulation before measurement of PSA levels representative of the nadir PSA level in any individual. In some cases, the nadir PSA may be determined or approximated by the measured concentration of PSA in a sample obtained from the patient at a time point of between three months and six months following a radical prostatectomy.

In some cases, the determination of the patient's prognosis for recurrence of prostate cancer and/or a course of treatment does not require measurement of a change in concentration of PSA measured in multiple samples as a function of time elapsed after the radical prostatectomy. That is, the determination made be based, at least in part, on one or more samples obtained contemporaneously or within a short time frame, wherein the determination does not require multiple samples obtained from the patient over a longer time frame. In some cases, the determination may be based at least in part on the concentration of PSA measured in a single sample obtained from the patient (“single sample” in this context refers to one or more samples collected at approximately the same time—e.g. with a single blood draw). In some cases, the determination may be based at least in part on the concentration of PSA measure in a plurality of samples obtained from the patient over a period of 1 hour, 2 hours, 3 hours, 4 hours, 5 hours, 6 hours, 12 hours, 24 hours, or 48 hours.

In some embodiments, the present invention provides methods of determining a patient's prognosis for recurrence of prostate cancer and/or determining a course of treatment for prostate cancer, following a radical prostatectomy, based at least in part, on the measured concentration of PSA in at least one sample obtained from the patient within 12 months following the radical prostatectomy. In some cases, the method comprises performing an assay on at least one sample obtained from the patient following the radical prostatectomy to determine a measure of the concentration of prostate specific antigen (PSA) in the at least one sample, wherein the at least one sample is obtained from the patient within 12 months following the radical prostatectomy, and determining the patient's prognosis for recurrence of prostate cancer and/or determining a course of treatment for prostate cancer following a radical prostatectomy based at least in part on the concentration of PSA measured in the at least one sample. In some cases, the samples are obtained from the patient within 9 months, 8 months, 7 months, 6 months, 5 months, 4 months, or 3 months or less of the radical prostatectomy. In some cases, the measure concentration of PSA is less than about 50 pg/mL, or another suitable range or level as described herein.

In some embodiments, a method of the present invention provides for determining a patient's prognosis for recurrence of prostate cancer and/or determining a course of treatment for prostate cancer following a radical prostatectomy based at least in part on an indication of the significant likelihood that the patient's prostate cancer will reoccur within 5 years. In some cases, the likelihood of a patient's prostate cancer reoccurring within a period of time case can be based, at least in part, on the measure of the concentration of PSA in at least one sample obtained from the patient following a radical prostatectomy. In some cases, the measured concentration of PSA used in the method is lower and/or is obtained from the patient in a shorter period of time following the radical prostatectomy as compared to typical conventional methods. In some cases, a concentration of PSA in at least one sample obtained from a patient following a radical prostatectomy greater than a threshold limit of no greater than about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, or about 20 pg/mL, indicates a significant likelihood that the patient's prostate cancer will reoccur within 5 years. In some cases, a measured concentration of PSA greater than a threshold limit of no greater than about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, or greater, in at least one sample obtained from a patient within about 1 month, about 2 months, about 3 months, about 4 months, about 5 months, about 6 months, about 7 months, about 8 months, about 9 months, about 10 months, about 11 months, about 12 months, about 15 months, about 18 months, about 2 years, or more, following a radical prostatectomy indicates a significant likelihood that the patient's prostate cancer will reoccur within 2 years, 3 years, 4 years, 5 years, 6 years, 7 years, 8 years, 9 years, 10 years, or more. In some cases, a measured concentration of PSA greater than about 3 pg/mL in at least one sample obtained from a patient at or within about 3 months, or at or within about 6 months, or at or within about 9 months following a radical prostatectomy indicates a significant likelihood that the patient's prostate cancer will reoccur within 5 years. In some cases, the significant likelihood indicates that the patient's chance of recurrence of prostate cancer is at least about 50%, about 60%, about 70%, about 75%, about 80%, about 85%, about 90%, about 95%, about 98%, about 99%, or about 99.5%, within the selected timeframe (e.g., 5 years).

Alternatively, in some embodiments, a method of the present invention provides for determining a patient's prognosis for recurrence of prostate cancer and/or determining a course of treatment for prostate cancer following a radical prostatectomy based at least in part on an indication of the significant likelihood that the patient's prostate cancer will not reoccur within 5 years. In some cases, the likelihood of a patient's prostate cancer not reoccurring within a period of time case can be based, at least in part, on the measure of the concentration of PSA in at least one sample obtained from the patient following a radical prostatectomy. In some cases, the measured concentration of PSA used in the method is lower and/or is obtained from the patient in a shorter period of time following the radical prostatectomy as compared to typical conventional methods. In some cases, a concentration of PSA in at least one sample obtained from a patient following a radical prostatectomy less than a threshold limit of no greater than about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, or about 20 pg/mL, indicates a significant likelihood that the patient's prostate cancer will not reoccur within 5 years. In some cases, a measured concentration of PSA less than a threshold limit of no greater than about 2 pg/mL, about 3 pg/mL, about 4 pg/mL, about 5 pg/mL, about 6 pg/mL, about 7 pg/mL, about 8 pg/mL, about 9 pg/mL, about 10 pg/mL, about 11 pg/mL, about 12 pg/mL, about 13 pg/mL, about 14 pg/mL, about 15 pg/mL, or about 20 pg/mL, in at least one sample obtained from a patient at or within about 1 month, about 2 months, about 3 months, about 4 months, about 5 months, about 6 months, about 7 months, about 8 months, about 9 months, about 10 months, about 11 months, about 12 months, about 15 months, about 18 months, about 2 years, or more, following a radical prostatectomy indicates a significant likelihood that the patient's prostate cancer will not reoccur within 2 years, 3 years, 4 years, 5 years, 6 years, 7 years, 8 years, 9 years, l0years, or more. In some cases, a measured concentration of PSA less than about 3 pg/mL in at least one sample obtained from a patient at about 3 months, or at or within about 6 months, or at or within about 9 months following a radical prostatectomy indicates a significant likelihood that the patient's prostate cancer not will reoccur within 5 years. In some cases, the significant likelihood of a patient's prostate cancer not reoccurring indicates that the patient's chance of recurrence of prostate cancer is less than about 50%, about 60%, about 70%, about 75%, about 80%, about 85%, about 90%, about 95%, about 98%, about 99%, or about 99.5%, within the selected time frame (e.g., 5 years).

In some cases, the method comprises performing an assay on at least one sample obtained from the patient following the radical pro statectomy to determine a measure of the concentration of prostate specific antigen (PSA) in the at least one sample, wherein the at least one sample is obtained from the patient within 12 months, 9 months, 8 months, 7 months, 6 months, 5 months, 4 months, or 3 months following the radical prostatectomy, and determining the patient's prognosis for recurrence of prostate cancer and/or determining a course of treatment for prostate cancer following a radical prostatectomy based at least in part on the concentration of PSA measured in the at least one sample. In some cases, a measured concentration of PSA greater than about 10 pg/mL in the at least one sample indicates a significant likelihood that the patient's prostate cancer will reoccur within 5 years.

In certain embodiments of the above methods, the measured concentration of PSA used to determine, at least in part, the prognosis and/or the method of treatment is less than about 100 pg/mL, less than about 90 pg/mL, less than about 80 pg/mL, less than about 70 pg/mL, less than about 60 pg/mL, less than about 50 pg/mL, less than about 40 pg/mL, less than about 30 pg/mL, less than about 20 pg/mL, less than about 15 pg/mL, less than about 10 pg/mL, less than about 9 pg/mL, less than about 8 pg/mL, less than about 7 pg/mL, less than about 6 pg/mL, less than about 5 pg/mL, less than about 4 pg/mL, less than about 3 pg/mL, less than about 2 pg/mL, or less than about 1 pg/mL. In some cases, the measured concentration is between about 1 pg/mL and about 100 pg/mL, between about 1 pg/mL and about 50 pg/mL, between about 1 pg/mL and about 20 pg/mL, between about 1 pg/mL and about 10 pg/mL, between about 5 pg/mL and about 15 pg/mL, or between about 1 pg/mL and about 5 pg/mL.

In embodiments where a plurality of samples is obtained from a patient, the samples may be obtained from a patient over any suitable period of time. In some cases, the sample is obtained from the patient at or less than about 1 week, about 2 weeks, about 1 month, about 2 months, about 3 months, about 4 months, about 5 months, about 6 months, about 7 months, about 8 months, about 9 months, about 10 months, about 11 months, about 12 months, about 18 months, about 2 years, or more, following the radical prostatectomy.

Any number of samples (e.g., one or more) may be obtained from the patient over the time period of sample collection. In some cases, at only a single time point is a sample(s) obtained from a patient following a radical prostatectomy and used in a method of the present invention. In some cases, samples collected at more than one sampling interval are obtained and analyzed, e.g. at least about 2, at least about 3, at least about 4, at least abut 5, at least about 6, at least about 7, at least about 8, at least about 9, at least about 10, at least about 12, at least about 15 or more collection times. In some cases, the number of samples obtained from the patient is between 2 and 20, between 5 and 15, or between 5 and 10. In some cases, the samples may be obtained at time intervals at about 1 week, about 2 weeks, about 1 month, about 2 months, about 3 months, about 4 months, about 5 months, about 6 months, about 7 months, about 8 months, about 9 months, about 10 months, about 11 months, about 12 months, or about 18 months, or more.

The sample(s) obtained from the patient may be from any suitable bodily source. In some cases, the samples are blood or blood products (e.g., whole blood, plasma, serum, etc.). In other cases, the samples may be urine, semen, or saliva samples. In some embodiments, the samples may be analyzed directly (e.g., without the need for extraction of PSA from the fluid sample) and/or with dilution (e.g., addition of a buffer or agent to the sample). Generally, each of the samples obtained from the patient is collected using substantially similar procedures (e.g., to ensure minimal variation between samples based on sample collection methods). Those of ordinary skill in the art will be aware of suitable systems and methods for obtaining a sample from a patient.

Those of ordinary skill in the art will be aware of suitable methods and systems for providing treatment to a patient who is determined to have a significant likelihood of recurrence of prostate cancer. Non-limiting examples of suitable treatments include surgery, radiation, chemotherapy, and/or immunotherapy.

As used herein, the term “patient” refers to a human. The patient may be male or female. In some cases, the patient is male. In some embodiments, a patient or subject may be under the care of a physician or other health care worker, including, but not limited to, someone who has consulted with, received advice from or received a prescription or other recommendation from a physician or other health care worker.

### Exemplary Assay Methods and Systems

Those of ordinary skill in the art will be aware of a variety of assay methods and systems that may be used in connection with the methods of the present invention. Generally, the methods employed have low limits of detection and/or limits of quantification as compared to bulk analysis techniques (e.g., ELISA methods). The use of assay methods that have low limits of detection and/or limits of quantification allows for correlations to be made between the various parameters discussed above and a method of treatment and/or diagnostic indication that may otherwise not be determinable and/or apparent. It will be understood by those of ordinary skill in the art, that while the terms “biomarker” and “biomarker molecule(s)” are used in this section to describe exemplary assay methods and systems, when used in connection with the present invention, in most embodiments, the biomarker and the biomarker molecule(s) are “PSA” and “PSA molecule(s),” respectively.

The terms “limit of detection” (or LOD) and “limit of quantification” (or LOQ) are given their ordinary meaning in the art. The LOD refers to the lowest analyte concentration likely to be reliably distinguished from background noise and at which detection is feasible. The LOD as used herein is defined as three standard deviations (SD) above background noise. The LOQ refers to the lowest concentration at which the analyte can not only be reliably detected but at which some predefined goals for bias and imprecision are met. Generally, as is used herein, the LOQ refers to the lowest concentration above the LOD wherein the coefficient of variation (CV) of the measured concentrations less than about 20%.

In some cases, an assay method employed has a limit of detection and/or a limit of quantification of less than about 500 pg/mL, 250 pg/mL, 100 pg/mL, 50 pg/mL, 40 pg/mL, 30 pg/mL, 20 pg/mL, 10 pg/mL 5 pg/mL, 4 pg/mL, 3 pg/mL, 2 pg/mL, 1 pg/mL, 0.8 pg/mL, 0.7 pg/mL, 0.6 pg/mL, 0.5 pg/mL, 0.4 pg/mL, 0.3 pg/mL, 0.2 pg/mL, 0.1 pg/mL, 0.05 pg/mL, 0.04 pg/mL, 0.02 pg/mL, 0.01 pg/mL, or less. In some cases, an assay method employed has a limit of quantification and/or a limit of detection between about 100 pg/mL and about 0.01 pg/mL, between about 50 pg/mL and about 0.02 pg/mL, between about 25 pg/mL and about 0.02 pg/mL, between about 10 pg/mL and about 0.02 pg/mL, between about 5 pg/mL and about 0.02 pg/mL, or between about 1 pg/mL and about 0.02 pg/mL. As will be understood by those of ordinary skill the art, the LOQ and/or LOD may differ for each assay method and/or each biomarker determined with the same assay. In some embodiments, the LOD of an assay employed for detecting of PSA is about equal to or less than 0.03 pg/mL, or about equal to or less than 0.02 pg/mL. In some embodiments, the LOQ for an assay employed for detecting PSA is equal to or less than 0.04 pg/mL, or equal to or less than 0.034 pg/mL.

In some embodiments, the concentration of biomarker molecules in the fluid sample that may be substantially accurately determined is less than about 5000 fM, less than about 3000 fM, less than about 2000 fM, less than about 1000 fM, less than about 500 fM, less than about 300 fM, less than about 200 fM, less than about 100 fM, less than about 50 fM, less than about 25 fM, less than about 10 fM, less than about 5 fM, less than about 2 fM, less than about 1 fM, less than about 0.5 fM, less than about 0.1 fM, or less. In some embodiments, the concentration of biomarker molecules in the fluid sample that may be substantially accurately determined is between about 5000 fM and about 0.1 fM, between about 3000 fM and about 0.1 fM, between about 1000 fM and about 0.1 fM, between about 1000 fM and about 1 fM, between about 100 fM and about 1 fM, between about 100 fM and about 0.1 fM, or the like. The concentration of analyte molecules or particles in a fluid sample may be considered to be substantially accurately determined if the measured concentration of the biomarker molecules in the fluid sample is within about 10% of the actual (e.g., true) concentration of the biomarker molecules in the fluid sample. In certain embodiments, the measured concentration of the biomarker molecules in the fluid sample may be within about 5%, within about 4%, within about 3%, within about 2%, within about 1%, within about 0.5%, within about 0.4%, within about 0.3%, within about 0.2% or within about 0.1%, of the actual concentration of the biomarker molecules in the fluid sample. In some cases, the measure of the concentration determined differs from the true (e.g., actual) concentration by no greater than about 20%, no greater than about 15%, no greater than 10%, no greater than 5%, no greater than 4%, no greater than 3%, no greater than 2%, no greater than 1%, or no greater than 0.5%. The accuracy of the assay method may be determined, in some embodiments, by determining the concentration of biomarker molecules in a fluid sample of a known concentration using the selected assay method.

In some embodiments, an assay method employs a step of spatially segregating biomarker molecules into a plurality of locations to facilitate detection/quantification, such that each location comprises/contains either zero or one or more biomarker molecules. Additionally, in some embodiments, the locations may be configured in a manner such that each location can be individually addressed. In some embodiments, a measure of the concentration of biomarker molecules in a fluid sample may be determined by detecting biomarker molecules immobilized with respect to a binding surface having affinity for at least one type of biomarker molecule. In certain embodiments the binding surface may form (e.g., a surface of a well/reaction vessel on a substrate) or be contained within (e.g., a surface of a capture object, such as a bead, contained within a well) one of a plurality of locations (e.g., a plurality of wells/reaction vessels) on a substrate (e.g., plate, dish, chip, optical fiber end, etc). At least a portion of the locations may be addressed and a measure indicative of the number/percentage/fraction of the locations containing at least one biomarker molecule may be made. In some cases, based upon the number/percentage/fraction, a measure of the concentration of biomarker molecules in the fluid sample may be determined. The measure of the concentration of biomarker molecules in the fluid sample may be determined by a digital analysis method/system optionally employing Poisson distribution adjustment and/or based at least in part on a measured intensity of a signal, as will be known to those of ordinary skill in the art. In some cases, the assay methods and/or systems may be automated.

Certain methods and systems which employ spatially segregating analyte molecules (e.g., biomarkers) are known in the art, and are described in U.S. Patent Application Publication No. US-2007-0259448 (Ser. No. 11/707,385), filed Feb. 16, 2007, entitled “METHODS AND ARRAYS FOR TARGET ANALYTE DETECTION AND DETERMINATION OF TARGET ANALYTE CONCENTRATION IN SOLUTION,” by Walt et al.; U.S. Patent Application Publication No. US-2007-0259385 (Ser. No. 11/707,383), filed Feb. 16, 2007, entitled “METHODS AND ARRAYS FOR DETECTING CELLS AND CELLULAR COMPONENTS IN SMALL DEFINED VOLUMES,” by Walt et al.; U.S. Patent Application Publication No. US-2007-0259381 (Ser. No. 11/707,384), filed Feb. 16, 2007, entitled “METHODS AND ARRAYS FOR TARGET ANALYTE DETECTION AND DETERMINATION OF REACTION COMPONENTS THAT AFFECT A REACTION,” by Walt et al.; International Patent Publication No. WO 2009/029073 (International Patent Application No. PCT/US2007/019184), filed Aug. 30, 2007, entitled “METHODS OF DETERMINING THE CONCENTRATION OF AN ANALYTE IN SOLUTION,” by Walt et al.; U.S. Patent Application Publication No. US-2010-0075862 (Ser. No. 12/236,484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al.; U.S. Patent Application Publication No. US-2010-00754072 (Ser. No. 12/236,486), filed Sep. 23, 2008, entitled “ULTRA-SENSITIVE DETECTION OF MOLECULES ON SINGLE MOLECULE ARRAYS,” by Duffy et al.; U.S. Patent Application Publication No. US-2010-0075439 (Ser. No. 12/236,488), filed Sep. 23, 2008, entitled “ULTRA-SENSITIVE DETECTION OF MOLECULES BY CAPTURE-AND-RELEASE USING REDUCING AGENTS FOLLOWED BY QUANTIFICATION,” by Duffy et al.; International Patent Publication No. WO2010/039179 (International Patent Application No. PCT/US2009/005248), filed Sep. 22, 2009, entitled “ULTRA-SENSITIVE DETECTION OF MOLECULES OR ENZYMES,” by Duffy et al.; U.S. Patent Application Publication No. US-2010-0075355 (Ser. No. 12/236,490), filed Sep. 23, 2008, entitled “ULTRA-SENSITIVE DETECTION OF ENZYMES BY CAPTURE-AND-RELEASE FOLLOWED BY QUANTIFICATION,” by Duffy et al.; U.S. Patent Application Publication No. US 2011-0212848 (Ser. No. 12/731,130), filed Mar. 24, 2010, entitled “ULTRA-SENSITIVE DETECTION OF MOLECULES OR PARTICLES USING BEADS OR OTHER CAPTURE OBJECTS,” by Duffy et al.; International Patent Publication No. WO2011/109364 (International Patent Application No. PCT/US2011/026645), filed Mar. 1, 2011, entitled “ULTRA-SENSITIVE DETECTION OF MOLECULES OR PARTICLES USING BEADS OR OTHER CAPTURE OBJECTS,” by Duffy et al.; International Patent Publication No. WO2011/109372 (International Patent Application No. PCT/US2011/026657), filed Mar. 1, 2011, entitled “ULTRA-SENSITIVE DETECTION OF MOLECULES USING DUAL DETECTION METHODS,” by Duffy et al.; U.S. Patent Application No. US 2011-0212462 (Ser. No. 12/731,135), filed Mar. 24, 2010, entitled “ULTRA-SENSITIVE DETECTION OF MOLECULES USING DUAL DETECTION METHODS,” by Duffy et al.; International Patent Publication No. WO2011/109379 (International Patent Application No. PCT/US2011/026665), filed Mar. 1, 2011, entitled “METHODS AND SYSTEMS FOR EXTENDING DYNAMIC RANGE IN ASSAYS FOR THE DETECTION OF MOLECULES OR PARTICLES,” by Rissin et al.; U.S. Patent Application No. US 2011-0212537 (Ser. No. 12/731,136), filed Mar. 24, 2010, entitled “METHODS AND SYSTEMS FOR EXTENDING DYNAMIC RANGE IN ASSAYS FOR THE DETECTION OF MOLECULES OR PARTICLES,” by Duffy et al.; U.S. Patent Application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.; U.S. Patent Application No. US 2011-0245097 (Ser. No. 13/037,987), filed Mar. 1, 2011, entitled “METHODS AND SYSTEMS FOR EXTENDING DYNAMIC RANGE IN ASSAYS FOR THE DETECTION OF MOLECULES OR PARTICLES,” by Rissin et al.; each herein incorporated by reference.

Additional details of exemplary, non-limiting assay methods which comprise one or more steps of spatially segregating biomarker molecules will now be described. In certain embodiments, a method for detection and/or quantifying biomarker molecules in a sample comprises immobilizing a plurality of biomarker molecules with respect to a plurality of capture objects (e.g., beads) that each include a binding surface having affinity for at least one type of biomarker. For example, the capture objects may comprise a plurality of beads comprising a plurality of capture components (e.g., an antibody having specific affinity for a biomarker of interest, etc.). At least some of the capture objects (e.g., at least some associated with at least one biomarker molecule) may be spatially separated/segregated into a plurality of locations, and at least some of the locations may be addressed/interrogated (e.g., using an imaging system). A measure of the concentration of biomarker molecules in the fluid sample may be determined based on the information received when addressing the locations (e.g., using the information received from the imaging system and/or processed using a computer implemented control system). In some cases, a measure of the concentration may be based at least in part on the number of locations determined to contain a capture object that is or was associated with at least one biomarker molecule. In other cases and/or under differing conditions, a measure of the concentration may be based at least in part on an intensity level of at least one signal indicative of the presence of a plurality of biomarker molecules and/or capture objects associated with a biomarker molecule at one or more of the addressed locations.

In some embodiments, the number/percentage/fraction of locations containing a capture object but not containing a biomarker molecule may also be determined and/or the number/percentage/fraction of locations not containing any capture object may also be determined. In such embodiments, a measure of the concentration of biomarker molecules in the fluid sample may be based at least in part on the ratio of the number of locations determined to contain a capture object associated with a biomarker molecule to the total number of locations determined to contain a capture object not associated with a biomarker molecule and/or a measure of the concentration of biomarker molecule in the fluid sample may be based at least in part on the ratio of the number of locations determined to contain a capture object associated with a biomarker molecule to the number of locations determined to not contain any capture objects. In yet other embodiments, a measure of the concentration of biomarker molecules in a fluid sample may be based at least in part on the ratio of the number of locations determined to contain a capture object and a biomarker molecule to the total number of locations addressed and/or analyzed.

In certain embodiments, at least some of the plurality of capture objects (e.g., at least some associated with at least one biomarker molecule) are spatially separated into a plurality of locations, for example, a plurality of reaction vessels in an array format. The plurality of reaction vessels may be formed in, on and/or of any suitable material, and in some cases, the reaction vessels can be sealed or may be formed upon the mating of a substrate with a sealing component, as discussed in more detail below. In certain embodiments, especially where quantization of the capture objects associated with at least one biomarker molecule is desired, the partitioning of the capture objects can be performed such that at least some (e.g., a statistically significant fraction; e.g., as described in International Patent Publication No. WO2011/109364 (International Patent Application No. PCT/US2011/026645), filed Mar. 1, 2011, entitled “ULTRA-SENSITIVE DETECTION OF MOLECULES OR PARTICLES USING BEADS OR OTHER CAPTURE OBJECTS,” by Duffy et al.) of the reaction vessels comprise at least one or, in certain cases, only one capture object associated with at least one biomarker molecule and at least some (e.g., a statistically significant fraction) of the reaction vessels comprise an capture object not associated with any biomarker molecules. The capture objects associated with at least one biomarker molecule may be quantified in certain embodiments, thereby allowing for the detection and/or quantification of biomarker molecules in the fluid sample by techniques described in more detail herein.

An exemplary assay method may proceed as follows. A sample fluid containing or suspected of containing biomarker molecules is provided. An assay consumable comprising a plurality of assay sites is exposed to the sample fluid. In some cases, the biomarker molecules are provided in a manner (e.g., at a concentration) such that a statistically significant fraction of the assay sites contain a single biomarker molecule and a statistically significant fraction of the assay sites do not contain any biomarker molecules. The assay sites may optionally be exposed to a variety of reagents (e.g., using a reagent loader) and or rinsed. The assay sites may then optionally be sealed and imaged (see, for example, U.S. Patent Application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.). The images are then analyzed (e.g., using a computer implemented control system) such that a measure of the concentration of the biomarker molecules in the fluid sample may be obtained, based at least in part, by determination of the number/fraction/percentage of assay sites which contain a biomarker molecule and/or the number/fraction/percentage of sites which do not contain any biomarker molecules. In some cases, the biomarker molecules are provided in a manner (e.g., at a concentration) such that at least some assay sites comprise more than one biomarker molecule. In such embodiments, a measure of the concentration of biomarker molecules in the fluid sample may be obtained at least in part on an intensity level of at least one signal indicative of the presence of a plurality of biomarker molecules at one or more of the assay sites

In some cases, the methods optionally comprise exposing the fluid sample to a plurality of capture objects, for example, beads. At least some of the biomarker molecules are immobilized with respect to a bead. In some cases, the biomarker molecules are provided in a manner (e.g., at a concentration) such that a statistically significant fraction of the beads associate with a single biomarker molecule and a statistically significant fraction of the beads do not associate with any biomarker molecules. At least some of the plurality of beads (e.g., those associated with a single biomarker molecule or not associated with any biomarker molecules) may then be spatially separated/segregated into a plurality of assay sites (e.g., of an assay consumable). The assay sites may optionally be exposed to a variety of reagents and/or rinsed. At least some of the assay sites may then be addressed to determine the number of assay sites containing a biomarker molecule. In some cases, the number of assay sites containing a bead not associated with a biomarker molecule, the number of assay sites not containing a bead and/or the total number of assay sites addressed may also be determined. Such determination(s) may then be used to determine a measure of the concentration of biomarker molecules in the fluid sample. In some cases, more than one biomarker molecule may associate with a bead and/or more than one bead may be present in an assay site. In some cases, the plurality biomarker molecules may be exposed to at least one additional reaction component prior to, concurrent with, and/or following spatially separating at least some of the biomarker molecules into a plurality of locations.

The biomarker molecules may be directly detected or indirectly detected. In the case of direct detection, a biomarker molecule may comprise a molecule or moiety that may be directly interrogated and/or detected (e.g., a fluorescent entity). In the case of indirect detection, an additional component is used for determining the presence of the biomarker molecule. For example, the biomarker molecules (e.g., optionally associated with a bead) may be exposed to at least one type of binding ligand. A “binding ligand,” is any molecule, particle, or the like which specifically binds to or otherwise specifically associates with a biomarker molecule to aid in the detection of the biomarker molecule. In certain embodiments, a binding ligand may be adapted to be directly detected (e.g., the binding ligand comprises a detectable molecule or moiety) or may be adapted to be indirectly detected (e.g., including a component that can convert a precursor labeling agent into a labeling agent). A component of a binding ligand may be adapted to be directly detected in embodiments where the component comprises a measurable property (e.g., a fluorescence emission, a color, etc.). A component of a binding ligand may facilitate indirect detection, for example, by converting a precursor labeling agent into a labeling agent (e.g., an agent that is detected in an assay). A “precursor labeling agent” is any molecule, particle, or the like, that can be converted to a labeling agent upon exposure to a suitable converting agent (e.g., an enzymatic component). A “labeling agent” is any molecule, particle, or the like, that facilitates detection, by acting as the detected entity, using a chosen detection technique. In some embodiments, the binding ligand may comprise an enzymatic component (e.g., horseradish peroxidase, beta-galactosidase, alkaline phosphatase, etc). A first type of binding ligand may or may not be used in conjunction with additional binding ligands (e.g., second type, etc.).

More than one type of binding may be employed in any given assay method, for example, a first type of binding ligand and a second type of binding ligand. In one example, the first type of binding ligand is able to associate with a first type of biomarker molecule and the second type of binding ligand is able to associate with the first binding ligand. In another example, both a first type of binding ligand and a second type of binding ligand may associate with the same or different epitopes of a single biomarker molecule, as described herein. In some embodiments, at least one binding ligand comprises an enzymatic component.

In some embodiments, a binding ligand and/or a biomarker may comprise an enzymatic component. The enzymatic component may convert a precursor labeling agent (e.g., an enzymatic substrate) into a labeling agent (e.g., a detectable product). A measure of the concentration of biomarker molecules in the fluid sample can then be determined based at least in part by determining the number of locations containing a labeling agent (e.g., by relating the number of locations containing a labeling agent to the number of locations containing a biomarker molecule (or number of capture objects associated with at least one biomarker molecule to total number of capture objects)). Non-limiting examples of enzymes or enzymatic components include horseradish peroxidase, beta-galactosidase, and alkaline phosphatase. Other non-limiting examples of systems or methods for detection include embodiments where nucleic acid precursors are replicated into multiple copies or converted to a nucleic acid that can be detected readily, such as the polymerase chain reaction (PCR), rolling circle amplification (RCA), ligation, Loop-Mediated Isothermal Amplification (LAMP), etc. Such systems and methods will be known to those of ordinary skill in the art, for example, as described in “DNA Amplification: Current Technologies and Applications,” Vadim Demidov et al., 2004.

Another exemplary embodiment of indirect detection is as follows. In some cases, the biomarker molecules may be exposed to a precursor labeling agent (e.g., enzymatic substrate) and the enzymatic substrate may be converted to a detectable product (e.g., fluorescent molecule) upon exposure to a biomarker molecule.

The assay methods and systems may employ a variety of different components, steps, and/or other aspects that will be known and understood by those of ordinary skill in the art. For example, a method may further comprise determining at least one background signal determination (e.g., and further comprising subtracting the background signal from other determinations), wash steps, and the like. In some cases, the assays or systems may include the use of at least one binding ligand, as described herein. In some cases, the measure of the concentration of biomarker molecules in a fluid sample is based at least in part on comparison of a measured parameter to a calibration curve. In some instances, the calibration curve is formed at least in part by determination at least one calibration factor, as described above.

In certain embodiments, solubilized, or suspended precursor labeling agents may be employed, wherein the precursor labeling agents are converted to labeling agents which are insoluble in the liquid and/or which become immobilized within/near the location (e.g., within the reaction vessel in which the labeling agent is formed). Such precursor labeling agents and labeling agents and their use is described in commonly owned U.S. Patent Application Publication No. US-2010-0075862 (Ser. No. 12/236484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al., incorporated herein by reference.

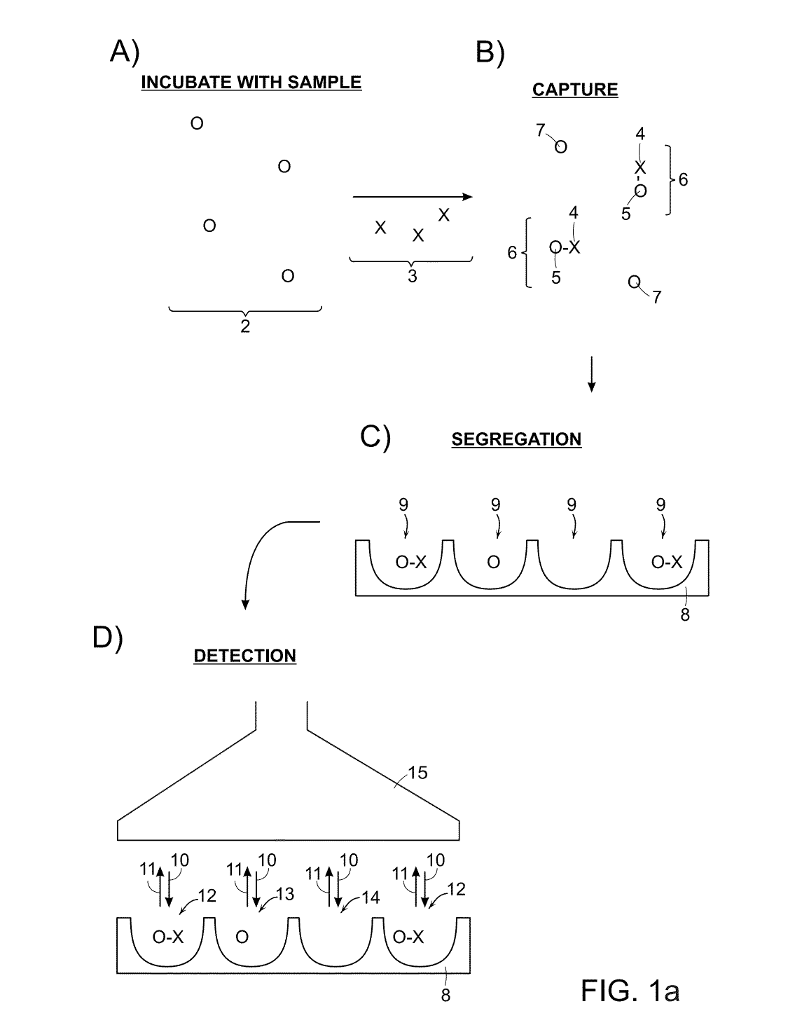

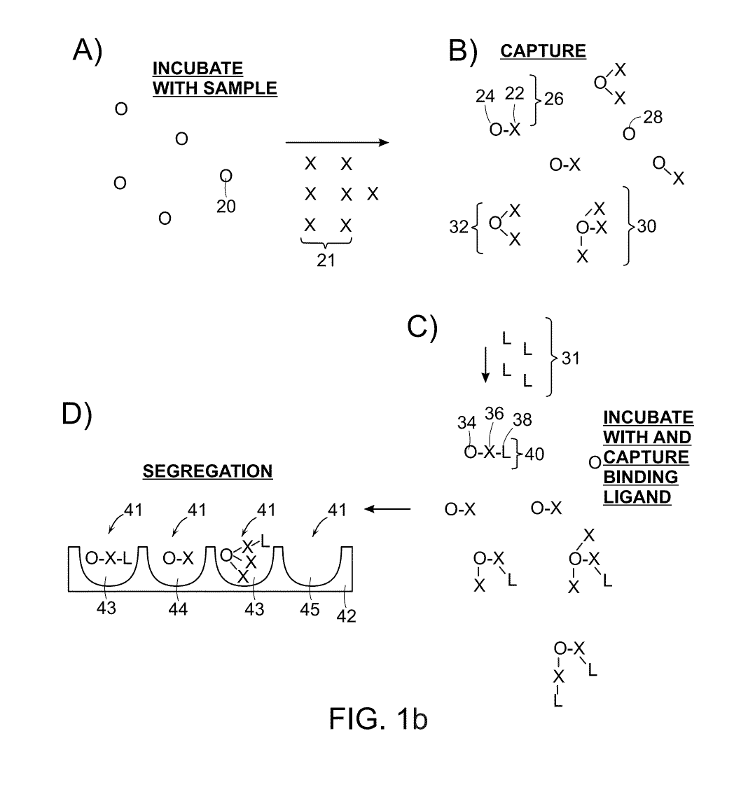

An exemplary embodiment of an assay method that may be used in certain embodiments of the invention is illustrated in FIG. 1a. A plurality of capture objects 2, are provided (step (A)). In this particular example, the plurality of capture objects comprises a plurality of beads. The beads are exposed to a fluid sample containing a plurality of biomarker molecules 3 (e.g., beads 2 are incubated with biomarker molecules 3). At least some of the biomarker molecules are immobilized with respect to a bead. In this example, the biomarker molecules are provided in a manner (e.g., at a concentration) such that a statistically significant fraction of the beads associate with a single biomarker molecule and a statistically significant fraction of the beads do not associate with any biomarker molecules. For example, as shown in step (B), biomarker molecule 4 is immobilized with respect to bead 5, thereby forming complex 6, whereas some beads 7 are not associated with any biomarker molecules. It should be understood, in some embodiments, more than one biomarker molecule may associate with at least some of the beads, as described herein. At least some of the plurality of beads (e.g., those associated with a single biomarker molecule or not associated with any biomarker molecules) may then be spatially separated/segregated into a plurality of locations. As shown in step (C), the plurality of locations is illustrated as substrate 8 comprising a plurality of wells/reaction vessels 9. In this example, each reaction vessel comprises either zero or one beads. At least some of the reaction vessels may then be addressed (e.g., optically or via other detection means) to determine the number of locations containing a biomarker molecule. For example, as shown in step (D), the plurality of reaction vessels are interrogated optically using light source 15, wherein each reaction vessel is exposed to electromagnetic radiation (represented by arrows 10) from light source 15. The light emitted (represented by arrows 11) from each reaction vessel is determined (and/or recorded) by detector 15 (in this example, housed in the same system as light source 15).

The number of reaction vessels containing a biomarker molecule (e.g., reaction vessels 12) is determined based on the light detected from the reaction vessels. In some cases, the number of reaction vessels containing a bead not associated with a biomarker molecule (e.g., reaction vessel 13), the number of wells not containing a bead (e.g., reaction vessel 14) and/or the total number of wells addressed may also be determined.

Such determination(s) may then be used to determine a measure of the concentration of biomarker molecules in the fluid sample.

A non-limiting example of an embodiment where a capture object is associated with more than one biomarker molecule is illustrated in FIG. 1b. A plurality of capture objects 20 are provided (step (A)). In this example, the plurality of capture objects comprises a plurality of beads. The plurality of beads is exposed to a fluid sample containing plurality of biomarker molecules 21 (e.g., beads 20 are incubated with biomarker molecules 21). At least some of the biomarker molecules are immobilized with respect to a bead. For example, as shown in step (B), biomarker molecule 22 is immobilized with respect to bead 24, thereby forming complex 26. Also illustrated is complex 30 comprising a bead immobilized with respect to three biomarker molecules and complex 32 comprising a bead immobilized with respect to two biomarker molecules. Additionally, in some cases, some of the beads may not associate with any biomarker molecules (e.g., bead 28). The plurality of beads from step (B) is exposed to a plurality of binding ligands 31. As shown in step (C), a binding ligand associates with some of the biomarker molecules immobilized with respect to a bead. For example, complex 40 comprises bead 34, biomarker molecule 36, and binding ligand 38. The binding ligands are provided in a manner such that a statistically significant fraction of the beads comprising at least one biomarker molecule become associated with at least one binding ligand (e.g., one, two, three, etc.) and a statistically significant fraction of the beads comprising at least one biomarker molecule do not become associated with any binding ligands. At least a portion of the plurality of beads from step (C) are then spatially separated into a plurality of locations. As shown in step (D), in this example, the locations comprise a plurality of reaction vessels 41 on a substrate 42. The plurality of reaction vessels may be exposed to the plurality of beads from step (C) such at each reaction vessel contains zero or one beads. The substrate may then be analyzed to determine the number of reaction vessels containing a binding ligand (e.g., reaction vessels 43), wherein in the number may be related to a measure of the concentration of biomarker molecules in the fluid sample. In some cases, the number of reaction vessels containing a bead and not containing a binding ligand (e.g., reaction vessel 44), the number of reaction vessels not containing a bead (e.g., reaction vessel 45), and/or the total number of reaction vessels addressed/analyzed may also be determined. Such determination(s) may then be used to determine a measure of the concentration of biomarker molecules in the fluid sample.

In some embodiments, a plurality of locations may be addressed and/or a plurality of capture objects and/or species/molecules/particles of interest may be detected substantially simultaneously. “Substantially simultaneously” when used in this context, refers to addressing/detection of the locations/capture objects/species/molecules/particles of interest at approximately the same time such that the time periods during which at least two locations/capture objects/species/molecules/particles of interest are addressed/detected overlap, as opposed to being sequentially addressed/detected, where they would not. Simultaneous addressing/detection can be accomplished by using various techniques, including optical techniques (e.g., CCD detector). Spatially segregating capture objects/species/molecules/particles into a plurality of discrete, resolvable locations, according to some embodiments facilitates substantially simultaneous detection by allowing multiple locations to be addressed substantially simultaneously. For example, for embodiments where individual species/molecules/particles are associated with capture objects that are spatially segregated with respect to the other capture objects into a plurality of discrete, separately resolvable locations during detection, substantially simultaneously addressing the plurality of discrete, separately resolvable locations permits individual capture objects, and thus individual species/molecules/particles (e.g., biomarker molecules) to be resolved. For example, in certain embodiments, individual molecules/particles of a plurality of molecules/particles are partitioned across a plurality of reaction vessels such that each reaction vessel contains zero or only one species/molecule/particle. In some cases, at least about 80%, at least about 85%, at least about 90%, at least about 95%, at least about 96%, at least about 97%, at least about 98%, at least about 99%, at least about 99.5% of all species/molecules/particles are spatially separated with respect to other species/molecules/particles during detection. A plurality of species/molecules/particles may be detected substantially simultaneously within a time period of less than about 1 second, less than about 500 milliseconds, less than about 100 milliseconds, less than about 50 milliseconds, less than about 10 milliseconds, less than about 1 millisecond, less than about 500 microseconds, less than about 100 microseconds, less than about 50 microseconds, less than about 10 microseconds, less than about 1 microsecond, less than about 0.5 microseconds, less than about 0.1 microseconds, or less than about 0.01 microseconds, less than about 0.001 microseconds, or less. In some embodiments, the plurality of species/molecules/particles may be detected substantially simultaneously within a time period of between about 100 microseconds and about 0.001 microseconds, between about 10 microseconds and about 0.01 microseconds, or less.

In some embodiments, the locations are optically interrogated. The locations exhibiting changes in their optical signature may be identified by a conventional optical train and optical detection system. Depending on the detected species (e.g., type of fluorescence entity, etc.) and the operative wavelengths, optical filters designed for a particular wavelength may be employed for optical interrogation of the locations. In embodiments where optical interrogation is used, the system may comprise more than one light source and/or a plurality of filters to adjust the wavelength and/or intensity of the light source. In some embodiments, the optical signal from a plurality of locations is captured using a CCD camera.

In some embodiments of the present invention, the plurality of reaction vessels may be sealed (e.g., after the introduction of the biomarker molecules, binding ligands, and/or precursor labeling agent), for example, through the mating of the second substrate and a sealing component. The sealing of the reaction vessels may be such that the contents of each reaction vessel cannot escape the reaction vessel during the remainder of the assay. In some cases, the reaction vessels may be sealed after the addition of the biomarker molecules and, optionally, at least one type of precursor labeling agent to facilitate detection of the biomarker molecules. For embodiments employing precursor labeling agents, by sealing the contents in some or each reaction vessel, a reaction to produce the detectable labeling agents can proceed within the sealed reaction vessels, thereby producing a detectable amount of labeling agents that is retained in the reaction vessel for detection purposes.

The plurality of locations may be formed may be formed using a variety of methods and/or materials. In some embodiments, the plurality of locations comprises a plurality of reaction vessels/wells on a substrate. In some cases, the plurality of reaction vessels is formed as an array of depressions on a first surface. In other cases, however, the plurality of reaction vessels may be formed by mating a sealing component comprising a plurality of depressions with a substrate that may either have a featureless surface or include depressions aligned with those on the sealing component. Any of the device components, for example, the substrate or sealing component, may be fabricated from a compliant material, e.g., an elastomeric polymer material, to aid in sealing. The surfaces may be or made to be hydrophobic or contain hydrophobic regions to minimize leakage of aqueous samples from the microwells. The reactions vessels, in certain embodiments, may be configured to receive and contain only a single capture object.

In some embodiments, the reaction vessels may all have approximately the same volume. In other embodiments, the reaction vessels may have differing volumes. The volume of each individual reaction vessel may be selected to be appropriate to facilitate any particular assay protocol. For example, in one set of embodiments where it is desirable to limit the number of capture objects used for biomarker capture contained in each vessel to a small number, the volume of the reaction vessels may range from attoliters or smaller to nanoliters or larger depending upon the nature of the capture objects, the detection technique and equipment employed, the number and density of the wells on the substrate and the expected concentration of capture objects in the fluid applied to the substrate containing the wells. In one embodiment, the size of the reaction vessel may be selected such only a single capture object used for biomarker capture can be fully contained within the reaction vessel (see, for example, U.S. Patent Application No. US 2011-0212848 (Ser. No. 12/731,130), filed Mar. 24, 2010, entitled “ULTRA-SENSITIVE DETECTION OF MOLECULES OR PARTICLES USING BEADS OR OTHER CAPTURE OBJECTS,” by Duffy et al.; International Patent Application Publication No. WO2011/109364 (International Patent Application No. PCT/US2011/026645), filed Mar. 1, 2011, entitled “ULTRA-SENSITIVE DETECTION OF MOLECULES OR PARTICLES USING BEADS OR OTHER CAPTURE OBJECTS ,” by Duffy et al., each herein incorporated by reference).

In some embodiments, the reaction vessels may have a volume between about 1 femtoliter and about 1 picoliter, between about 1 femtoliters and about 100 femtoliters, between about 10 attoliters and about 100 picoliters, between about 1 picoliter and about 100 picoliters, between about 1 femtoliter and about 1 picoliter, or between about 30 femtoliters and about 60 femtoliters. In some cases, the reaction vessels have a volume of less than about 1 picoliter, less than about 500 femtoliters, less than about 100 femtoliters, less than about 50 femtoliters, or less than about 1 femtoliter. In some cases, the reaction vessels have a volume of about 10 femtoliters, about 20 femtoliters, about 30 femtoliters, about 40 femtoliters, about 50 femtoliters, about 60 femtoliters, about 70 femtoliters, about 80 femtoliters, about 90 femtoliters, or about 100 femtoliters.

The total number of locations and/or density of the locations employed in an assay (e.g., the number/density of reaction vessels in an array) can depend on the composition and end use of the array. For example, the number of reaction vessels employed may depend on the number of types of biomarker molecule and/or binding ligand employed, the suspected concentration range of the assay, the method of detection, the size of the capture objects, the type of detection entity (e.g., free labeling agent in solution, precipitating labeling agent, etc.). Arrays containing from about 2 to many billions of reaction vessels (or total number of reaction vessels) can be made by utilizing a variety of techniques and materials. Increasing the number of reaction vessels in the array can be used to increase the dynamic range of an assay or to allow multiple samples or multiple types of biomarker molecules to be assayed in parallel. The array may comprise between one thousand and one million reaction vessels per sample to be analyzed. In some cases, the array comprises greater than one million reaction vessels. In some embodiments, the array comprises between about 1,000 and about 50,000, between about 1,000 and about 1,000,000, between about 1,000 and about 10,000, between about 10,000 and about 100,000, between about 100,000 and about 1,000,000, between about 100,000 and about 500,000, between about 1,000 and about 100,000, between about 50,000 and about 100,000, between about 20,000 and about 80,000, between about 30,000 and about 70,000, between about 40,000 and about 60,000 reaction vessels. In some embodiments, the array comprises about 10,000, about 20,000, about 50,000, about 100,000, about 150,000, about 200,000, about 300,000, about 500,000, about 1,000,000, or more, reaction vessels.

The array of reaction vessels may be arranged on a substantially planar surface or in a non-planar three-dimensional arrangement. The reaction vessels may be arrayed in a regular pattern or may be randomly distributed. In a specific embodiment, the array is a regular pattern of sites on a substantially planar surface permitting the sites to be addressed in the X-Y coordinate plane.

In some embodiments, the reaction vessels are formed in a solid material. As will be appreciated by those in the art, the number of potentially suitable materials in which the reaction vessels can be formed is very large, and includes, but is not limited to, glass (including modified and/or functionalized glass), plastics (including acrylics, polystyrene and copolymers of styrene and other materials, polypropylene, polyethylene, polybutylene, polyurethanes, cyclic olefin copolymer (COC), cyclic olefin polymer (COP), Teflon®, polysaccharides, nylon or nitrocellulose, etc.), elastomers (such as poly(dimethyl siloxane) and poly urethanes), composite materials, ceramics, silica or silica-based materials (including silicon and modified silicon), carbon, metals, optical fiber bundles, or the like. In general, the substrate material may be selected to allow for optical detection without appreciable autofluorescence. In certain embodiments, the reaction vessels may be formed in a flexible material.

A reaction vessel in a surface (e.g., substrate or sealing component) may be formed using a variety of techniques known in the art, including, but not limited to, photolithography, stamping techniques, molding techniques, etching techniques, or the like. As will be appreciated by those of the ordinary skill in the art, the technique used can depend on the composition and shape of the supporting material and the size and number of reaction vessels.

In a particular embodiment, an array of reaction vessels is formed by creating microwells on one end of a fiber optic bundle and utilizing a planar compliant surface as a sealing component. In certain such embodiments, an array of reaction vessels in the end of a fiber optic bundle may be formed as follows. First, an array of microwells is etched into the end of a polished fiber optic bundle. Techniques and materials for forming and etching a fiber optic bundle are known to those of ordinary skill in the art. For example, the diameter of the optical fibers, the presence, size and composition of core and cladding regions of the fiber, and the depth and specificity of the etch may be varied by the etching technique chosen so that microwells of the desired volume may be formed. In certain embodiments, the etching process creates microwells by preferentially etching the core material of the individual glass fibers in the bundle such that each well is approximately aligned with a single fiber and isolated from adjacent wells by the cladding material. Potential advantages of the fiber optic array format is that it can produce thousands to millions of reaction vessels without complicated microfabrication procedures and that it can provide the ability to observe and optically address many reaction vessels simultaneously.

Each microwell may be aligned with an optical fiber in the bundle so that the fiber optic bundle can carry both excitation and emission light to and from the wells, enabling remote interrogation of the well contents. Further, an array of optical fibers may provide the capability for simultaneous or non-simultaneous excitation of molecules in adjacent vessels, without signal “cross-talk” between fibers. That is, excitation light transmitted in one fiber does not escape to a neighboring fiber. Alternatively, the equivalent structures of a plurality of reaction vessels may be fabricated using other methods and materials that do not utilize the ends of an optical fiber bundle as a substrate. For example, the array may be a spotted, printed or photolithographically fabricated substrate produced by techniques known in the art; see for example WO95/25116; WO95/35505; PCT US98/09163; U.S. Pat. Nos. 5,700,637, 5,807,522, 5,445,934, 6,406,845, and 6,482,593. In some cases, the array may be produced using molding, embossing, and/or etching techniques as will be known to those of ordinary skill in the art.

In some embodiments, the plurality of locations may not comprise a plurality of reaction vessels/wells. For example, in embodiments where capture objects are employed, a patterned substantially planar surface may be employed and the patterned areas form a plurality of locations. In some cases, the patterned areas may comprise substantially hydrophilic surfaces which are substantially surrounded by substantially hydrophobic surfaces. In certain embodiments, a plurality of capture objects (e.g., beads) may be substantially surrounded by a substantially hydrophilic medium (e.g., comprising water), and the beads may be exposed to the patterned surface such that the beads associate in the patterned areas (e.g., the hydrophilic locations on the surface), thereby spatially segregating the plurality of beads. For example, in one such embodiment, a substrate may be or include a gel or other material able to provide a sufficient barrier to mass transport (e.g., convective and/or diffusional barrier) to prevent capture objects used for biomarker capture and/or precursor labeling agent and/or labeling agent from moving from one location on or in the material to another location so as to cause interference or cross-talk between spatial locations containing different capture objects during the time frame required to address the locations and complete the assay. For example, in one embodiment, a plurality of capture objects is spatially separated by dispersing the capture objects on and/or in a hydrogel material. In some cases, a precursor labeling agent may be already present in the hydrogel, thereby facilitating development of a local concentration of the labeling agent (e.g., upon exposure to a binding ligand or biomarker molecule carrying an enzymatic component). As still yet another embodiment, the capture objects may be confined in one or more capillaries. In some cases, the plurality of capture objects may be absorbed or localized on a porous or fibrous substrate, for example, filter paper. In some embodiments, the capture objects may be spatially segregated on a uniform surface (e.g., a planar surface), and the capture objects may be detected using precursor labeling agents which are converted to substantially insoluble or precipitating labeling agents that remain localized at or near the location of where the corresponding capture object is localized. The use of such substantially insoluble or precipitating labeling agents is described herein. In some cases, single biomarker molecules may be spatially segregated into a plurality of droplets. That is, single biomarker molecules may be substantially contained in a droplet containing a first fluid. The droplet may be substantially surrounded by a second fluid, wherein the second fluid is substantially immiscible with the first fluid.

In some embodiments, during the assay, at least one washing step may be carried out. In certain embodiments, the wash solution is selected so that it does not cause appreciable change to the configuration of the capture objects and/or biomarker molecules and/or does not disrupt any specific binding interaction between at least two components of the assay (e.g., a capture component and a biomarker molecule). In other cases, the wash solution may be a solution that is selected to chemically interact with one or more assay components. As will be understood by those of ordinary skill in the art, a wash step may be performed at any appropriate time point during the inventive methods.

For example, a plurality of capture objects may be washed after exposing the capture objects to one or more solutions comprising biomarker molecules, binding ligands, precursor labeling agents, or the like. As another example, following immobilization of the biomarker molecules with respect to a plurality of capture objects, the plurality of capture objects may be subjected to a washing step thereby removing any biomarker molecules not specifically immobilized with respect to a capture object.

Other assay methods in addition to those described herein are known in the art and may be used in connection with the inventive methods. For example, various analyzers are commercially available for the determination of the concentration of biomarkers. The assay methods employed should meet the algorithm requirements for LOD and LOQ.

U.S. Provisional Application No. 61/495,355, filed Jun. 9, 2011, by David Wilson et al., and entitled “METHODS OF DETERMINING A PATIENT'S PROGNOSIS FOR RECURRENCE OF PROSTATE CANCER AND/OR DETERMINING A COURSE OF TREATMENT FOR PROSTATE CANCER FOLLOWING A RADICAL PROSTATECTOMY,” is herein incorporated by reference.

The following examples are included to demonstrate various features of the invention. Those of ordinary skill in the art should, in light of the present disclosure, will appreciate that many changes can be made in the specific embodiments which are disclosed while still obtaining a like or similar result without departing from the scope of the invention as defined by the appended claims. Accordingly, the following examples are intended only to illustrate certain features of the present invention, but do not necessarily exemplify the full scope of the invention.

### EXAMPLE 1

Measuring prostate specific antigen (PSA) in prostate cancer patients following radical prostatectomy (RP) has been limited by the sensitivity of available assays. Because radical prostatectomy removes the tissue responsible for producing PSA, post-RP PSA levels are typically undetectable with typical current assay methods. However, more sensitive determination of post-RP PSA status has the potential to improve recurrence prognosis, selection for secondary treatment, and effectiveness of salvage treatment from more timely intervention. This example describes the analytical performance of a digital immunoassay with two logs greater sensitivity than typical current PSA assays. Utility of the test for precise measurement of PSA status in post-RP patients is also reported.

Method: Reagents were developed for a paramagnetic bead-based ELISA for use in high-density single molecule arrays. Individual anti-PSA capture-beads with immunocomplexes and associated enzyme labels (β-galactosidase) were singulated within the microarrays and interrogated for presence of enzyme label. Wells containing an enzyme immunocomplex converted substrate reporter molecules to a fluorescent product, which became concentrated in the small microwell volume. This permitted imaging of wells containing single molecules of label with a CCD camera. Poisson statistics predict that each well contains either one PSA molecule or no PSA molecules when the ratio of bound PSA per bead is much less than one. Raw signal was recorded as “% active wells”, which was converted to “average enzymes/bead” to correct for non-Poisson behavior at higher PSA concentrations. The output was related to a standard curve and converted to a PSA concentration of the sample. Analytical performance of the assay was characterized, its accuracy was compared with a commercially available test, and longitudinal serum samples from 30 post-RP patients were analyzed.

For description of various details associated with this assay, see, Example 4. Additional details are described in, for example, U.S. Patent Application No. US 2011-0212848 (Ser. No. 12/731,130), filed Mar. 24, 2010, entitled “ULTRA-SENSITIVE DETECTION OF MOLECULES OR PARTICLES USING BEADS OR OTHER CAPTURE OBJECTS,” by Duffy et al.; International Patent Publication No. WO2011/109364 (International Patent Application No. PCT/US2011/026645), filed Mar. 1, 2011, entitled “ULTRA-SENSITIVE DETECTION OF MOLECULES OR PARTICLES USING BEADS OR OTHER CAPTURE OBJECTS,” by Duffy et al.; International Patent Publication No. WO2011/109372 (International Patent Application No. PCT/US2011/026657), filed Mar. 1, 2011, entitled “ULTRA-SENSITIVE DETECTION OF MOLECULES USING DUAL DETECTION METHODS,” by Duffy et al.; U.S. Patent Application No. US 2011-0212462 (Ser. No. 12/731,135), filed Mar. 24, 2010, entitled “ULTRA-SENSITIVE DETECTION OF MOLECULES USING DUAL DETECTION METHODS,” by Duffy et al.; International Patent Publication No. WO2011/109379 (International Patent Application No. PCT/US2011/026665), filed Mar. 1, 2011, entitled “METHODS AND SYSTEMS FOR EXTENDING DYNAMIC RANGE IN ASSAYS FOR THE DETECTION OF MOLECULES OR PARTICLES,” by Rissin et al.; U.S. Patent Application No. US 2011-0212537 (Ser. No. 12/731,136), filed Mar. 24, 2010, entitled “METHODS AND SYSTEMS FOR EXTENDING DYNAMIC RANGE IN ASSAYS FOR THE DETECTION OF MOLECULES OR PARTICLES,” by Duffy et al.; U.S. patent application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.; U.S. Patent Application No. US 2011-0245097 (Ser. No. 13/037,987), filed Mar. 1, 2011, entitled “METHODS AND SYSTEMS FOR EXTENDING DYNAMIC RANGE IN ASSAYS FOR THE DETECTION OF MOLECULES OR PARTICLES,” by Rissin et al.; each herein incorporated by reference.

Results: Limit of Detection (3SD method) for PSA was estimated as 0.000028 ng/mL (0.028 pg/mL) across 20 experiments. Limit of Quantification, LOQ (PSA concentration at 20% measurement variation) for PSA was estimated over a six-week period as 0.000035 ng/mL (0.035 pg/mL). Reproducibility was characterized over a 10-day period with a panel of four prepared samples, the lowest of which was near the LOQ. Total CVs (including within run, between run, between day) were 8.8, 8.4, 9.9, and 18.3% at PSA concentrations of 51.5, 5.07, 0.99 and 0.04 pg/mL respectively. Linearity was confirmed across the calibration range (0-100 pg/mL) per CLSI EP6-A, and recovery in the absence and presence of endogenous interferences was within 10% of expected. Accuracy was assessed by comparison to a commercially available equimolar PSA method standardized with WHO reference material. Linear regression statistics across 48 serum samples using this assay=1.01(Centaur)+0.0025, R²=0.970 (standard error 0.53, Centaur range 0.41-13.56 ng/mL). All post-RP samples tested were well above the assay LOQ. PSA nadir values following surgery were strongly predictive of five-year biochemical recurrence-free survival.

Conclusion: The assay demonstrated a robust two-log advance in measurement sensitivity relative to current ultrasensitive assays, and the analytical performance required for a new enabling tool for highly accurate assessment of post-RP PSA status.

### EXAMPLE 2

The example describes a PSA assay based on a digital immunoassay technology utilizing high-density arrays of femtoliter-volume wells and single molecule counting. Detailed analytical validation data is provided. The assay has a LOQ of less than 0.00005 ng/mL, and reliably quantified serum PSA in post radical prostatectomy patients tested. The test can potentially be used to measure PSA in patients following primary and secondary therapy, improve biochemical recurrence (BCR) risk stratification, and better inform clinical decisions for use of secondary treatment.

Materials and Methods

For more information regarding the assay method, see Example 4.

SINGLE MOLECULE ARRAYS: Single molecule array technology involves performing a paramagnetic bead-based assay, followed by isolation of individual capture beads in arrays of femtoliter-sized reaction wells. Singulation of capture beads within microwells permits buildup of fluorescent product from an enzyme label, such that signal from a single immunocomplex can be readily detected with a CCD camera. At very low PSA concentrations, Poisson statistics predict that bead-containing microwells in the array will contain either a single labeled PSA molecule or no PSA molecules, resulting in a digital signal. With single-molecule sensitivity, concentrations of labeling reagents can be lowered, resulting in reduced non-specific background. This effect enables high signal:background ratios at extremely low analyte concentrations.

Arrays of femtoliter-volume wells were prepared. In brief, the ends of bundles of 50,000 optical fibers were polished with diamond lapping films. One end of each bundle was etched in mild acid solution. Differential etch rates of the optical fiber core and cladding glass of the bundles causes 4.5 μm diameter, 3.5 μm deep wells to be formed, giving an array of 50,000 microwells across the bundle. Optical fiber arrays were mounted in linear groups of eight within glass holders for bead loading and imaging. Groups of eight arrays were chosen to correspond with microtiter plate columns of eight wells, which were used as rinse troughs for washing array surfaces following bead loading.

REAGENTS: Three reagents were developed: paramagnetic PSA capture beads, biotinylated detector, and a streptavidin:β-galactosidase (SβG) conjugate. The capture beads were comprised of a monoclonal anti-PSA antibody (BiosPacific) directed to amino acid residues 158-163. The antibody was covalently attached by standard coupling chemistry to 2.7 μm carboxy paramagnetic microbeads (Varian). The antibody-coated beads were diluted to a concentration of 5×10⁶ beads/mL in Tris with a surfactant and BSA. Biotinylated detector reagent was comprised of a monoclonal anti PSA antibody (BiosPacific) directed to amino acid residues 3-11. The antibody was biotinylated using standard methods and diluted to a concentration of 0.15 μg/ml in a PBS diluent containing a surfactant and newborn calf serum, NCS (PBS/NCS). SβG was prepared by covalent conjugation of purified streptavidin (Thermo Scientific) and βG (Sigma) using standard coupling chemistry. For assay, aliquots of a concentrated SβG stock were diluted to 15 pM in PBS/NCS with 1 mM MgCl₂. /

CALIBRATION: The assay was calibrated using WHO 90:10 PSA standards (National Institute for Biological Standards and Control). A stock PSA solution was prepared by dilution to 2 mg/mL in PBS/Tween-20. Assay calibrators were prepared by dilution of the stock solution in 25% NCS/PBS with Tween-20, EDTA and ProClin 300. Calibrators were prepared in a serial series from 0.1 to 100 pg/ml to emphasize quantification accuracy below 100 pg/mL. Recovery studies indicated that use of NCS as a calibrator base gave equivalent accuracy to human serum (not shown).

ASSAY METHODS: Bead-sample incubations and labeling of immunocomplexes in conical 96 well plates (Axygen) were conducted. In brief, the assay was performed in three steps, starting with analyte capture, incubation with biotinylated detector, and labeling of the immunocomplexes with SβG. Following assay and bead collection with a magnet, beads were loaded onto the arrays for imaging in a loading buffer comprised of PBS and 0.01% Tween-20, MgCl₂, and sucrose.

ARRAY IMAGING: Beads from the assay were loaded onto the arrays. Wells containing beads with labeled PSA were visualized by the hydrolysis of enzyme substrate (resorufin β-D-galactopyranoside, RGP, Invitrogen) by βG into fluorescent product. RGP was introduced to the wells during sealing of the arrays with a silicon gasket. Enzyme-containing wells were imaged by fluorescence microscope fitted with a CCD camera. The images were analyzed to determine the average number of label enzymes/bead (AEB). At <70% active beads relative to total beads (low PSA), the signal output is a count of active beads corrected for a low statistical probability of multiple enzymes/bead (29). At >70% active beads (higher PSA), the probability of multiple enzymes/bead increases, and average fluorescence of the wells is converted to AEB based on the average intensities of wells containing single enzymes determined at lower concentrations. The AEB unit thus works continuously across the digital and analog realms.

RP PATIENTS: Retrospective longitudinal serum samples from 20 non-recurring (BCR-free for at least five years) and 13 biochemically recurring RP patients were obtained under IRB approval and de-identified. All subjects had undergone radical retropubic prostatectomy without neo-adjuvant hormonal therapy. Targeted longitudinal sampling was a serum draw between 3 and 6 months after RP (nadir PSA), followed 3-6 months later by two subsequent draws separated by 3-6 months. Patients with positive lymph nodes at the time of surgery were excluded, as were patients who received neo-adjuvant or adjuvant therapy prior to BCR. BCR was defined as two consecutive PSA levels 0.2 ng/mL (200 pg/mL) after the initial collected sample, or secondary treatment.

SAMPLE HANDLING AND MEASUREMENT OF SERUM PSA: Specimens were stored at -70° C. until assayed. To limit effects of potential interferences, thawed samples were centrifuged at 9000g for 3-5 minutes and pre-diluted 1:4 in a diluent containing PBS with 0.01% Tween-20, heterophilic blocker, and EDTA prior to assay. Samples and calibrators were assayed in triplicate, and serial patient samples were tested within a single plate. Specimens above the highest calibrator were diluted 100-fold with the zero calibrator and re-assayed.

Results

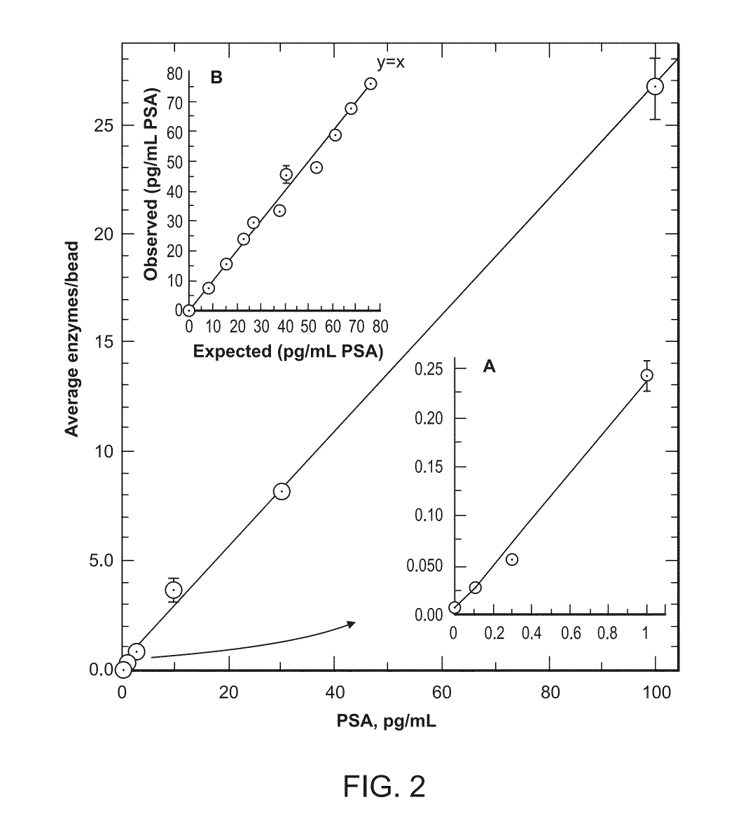

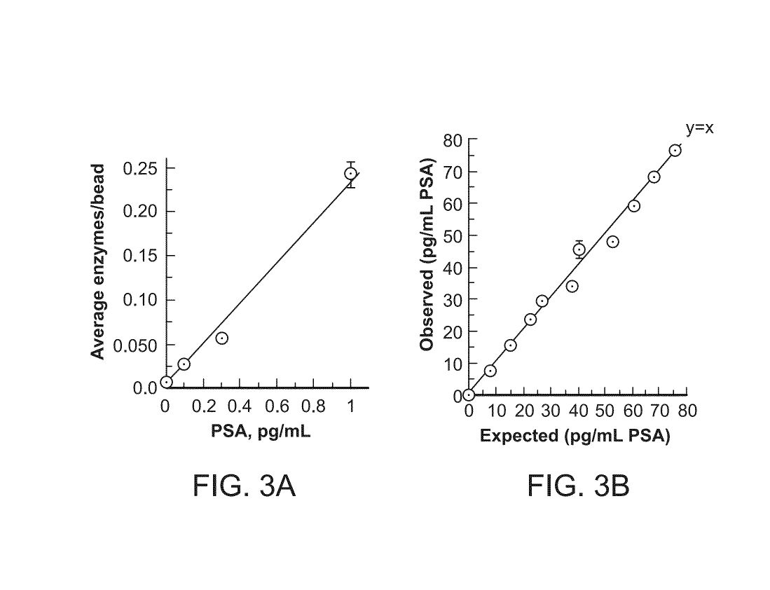

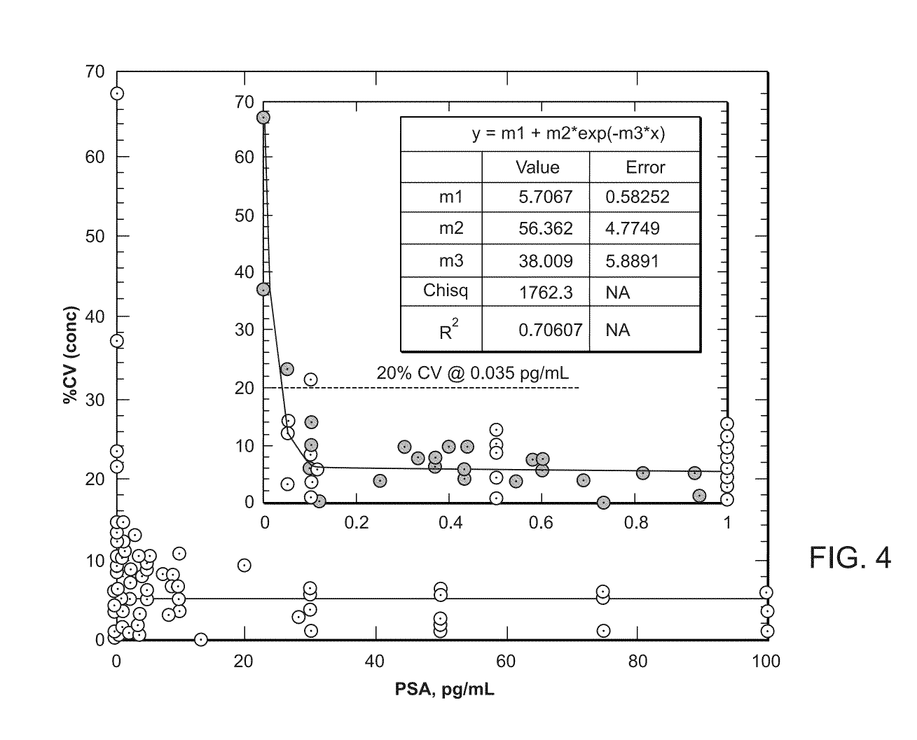

DOSE-RESPONSE, LINEARITY, AND RECOVERY: FIG. 2 shows a representative dose-response across three and a half logs of range. The assay demonstrated a highly linear response (R² 0.999). M a study of 20 calibration curves over 10 days, the mean signal to noise ratio at 0.1 pg/mL was 4.33 (SD 0.76). Linearity, conducted with guidance from CLSI protocol EP6-A (31), was evaluated with admixtures of female serum exhibiting relatively high and very low PSA levels (FIG. 3b). Linear (depicted) and 3^{rd} order polynomial fit goodness was virtually identical (R² 0.988 and 0.990 respectively). Percent deviation from linearity between the two models was within 5% across the range. Recovery of spiked PSA from serum in the absence and presence of supplemented high levels of potential endogenous interferences (20 mg/dL bilirubin, 1000 mg/dL triglycerides, 12 g/dL protein, 20 mg/dL hemoglobin) was within 10% of expected.

SENSITIVITY: Analytical Limit of Detection (LOD) was estimated as three standard deviations above background. LOD was calculated for each of 20 calibration runs from triplicate measurements of the zero calibrator and the lowest PS A-containing calibrator (0.1 pg/mL). The mean LOD was 0.028 pg/mL (SD 0.039 pg/mL). The LOQ was estimated from sample replicate CVs (n=3) obtained across the assay range over six weeks. The resulting CV profile is depicted in FIG. 4. The replicates were obtained from repeated measurement of assay calibrators, controls, and female serum. CVs for the different sample types were not statistically different. The estimated LOQ was the concentration of PSA corresponding to a 20% CV. From the equation of the power fit, the LOQ was calculated as 0.0352 pg/mL (standard error 0.0340-0.0387 pg/mL).

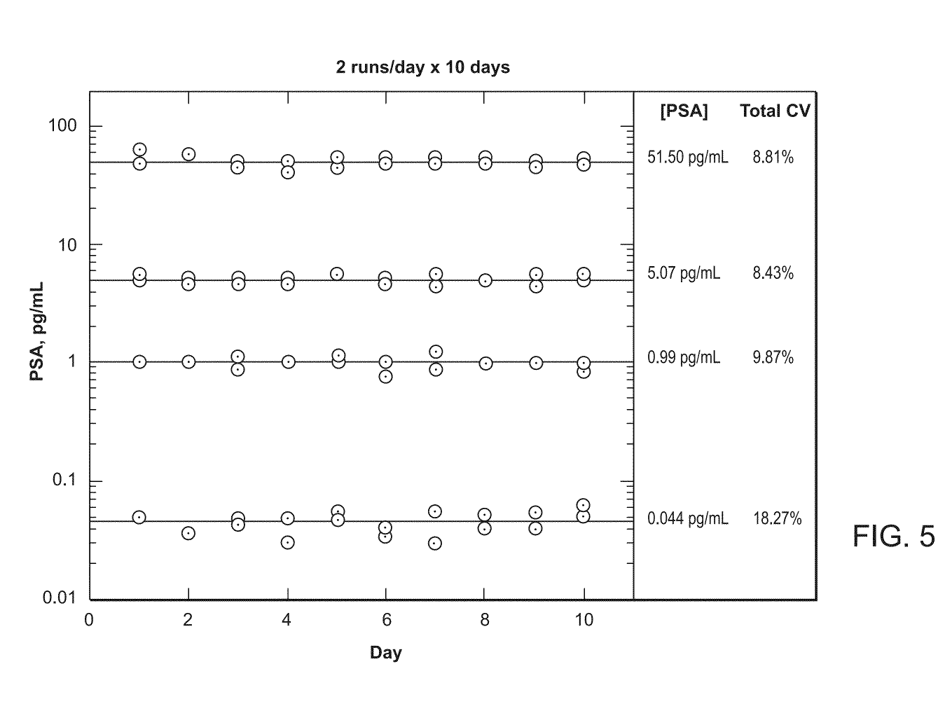

REPRODUCIBILITY: Reproducibility was assessed with guidance from CLSI EP5-A2 (32). Four samples, consisting of 90:10 PSA spiked into 25% NCS, were assayed in triplicate in each of two separate runs per day for 10 days (n=60 for each sample). The lowest sample was prepared near the estimated LOQ (0.035 pg/mL). Because each reportable result is based on triplicate measurements, this protocol gave two results/day for each sample. The plate map was configured such that each PSA result spanned multiple columns, which meant that replicates included variation from different groups of arrays. PSA results were calculated from within-plate calibration curves. Thus, the overall study comprehended array processing variation, calibration variation, and within-run, between-run, and day-to-day variation. The results of the study are depicted in FIG. 5. Total CVs across all variation sources were less than 10% from 1 to 52 pg/mL PSA. The total CV for the 0.04 pg/mL sample was 18.27%, consistent with the LOQ estimate (20% CV at 0.035 pg/mL).

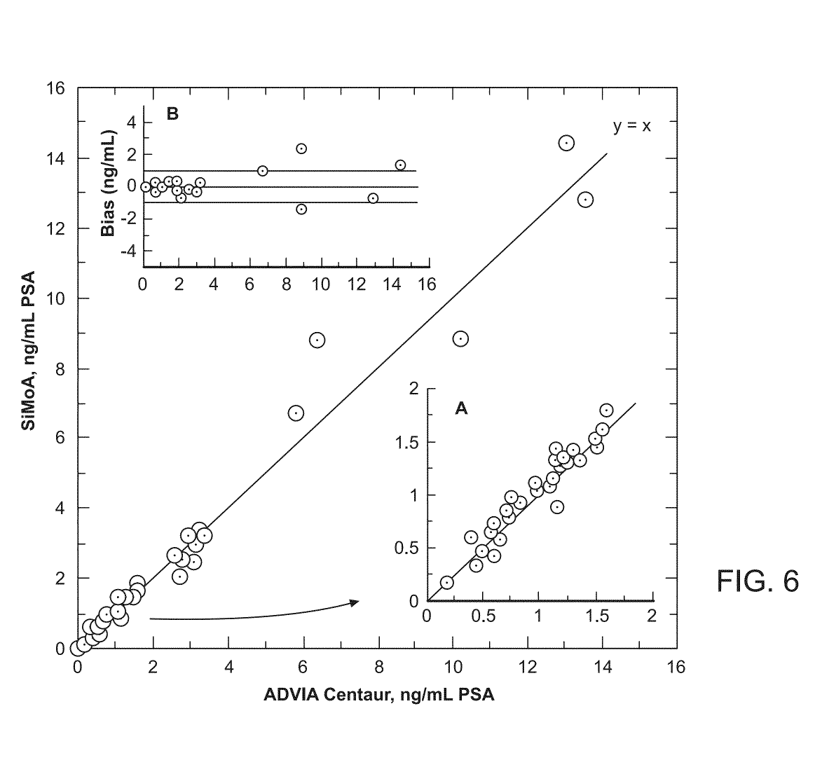

ACCURACY: Accuracy was assessed by comparison to a commercially available equimolar PS A method standardized with WHO reference material. 40 serum samples from normal males and eight serum samples from RP patients with PSA levels high enough for measurement in the comparator method (ADVIA Centaur, Siemens; LOD 0.1 ng/mL) were assayed with both methods (FIG. 6). All samples were diluted 100-fold prior to testing. The assays exhibited excellent agreement with no significant bias throughout the range of results (0.17 to >13 ng/mL, mean bias 0.024 ng/mL).

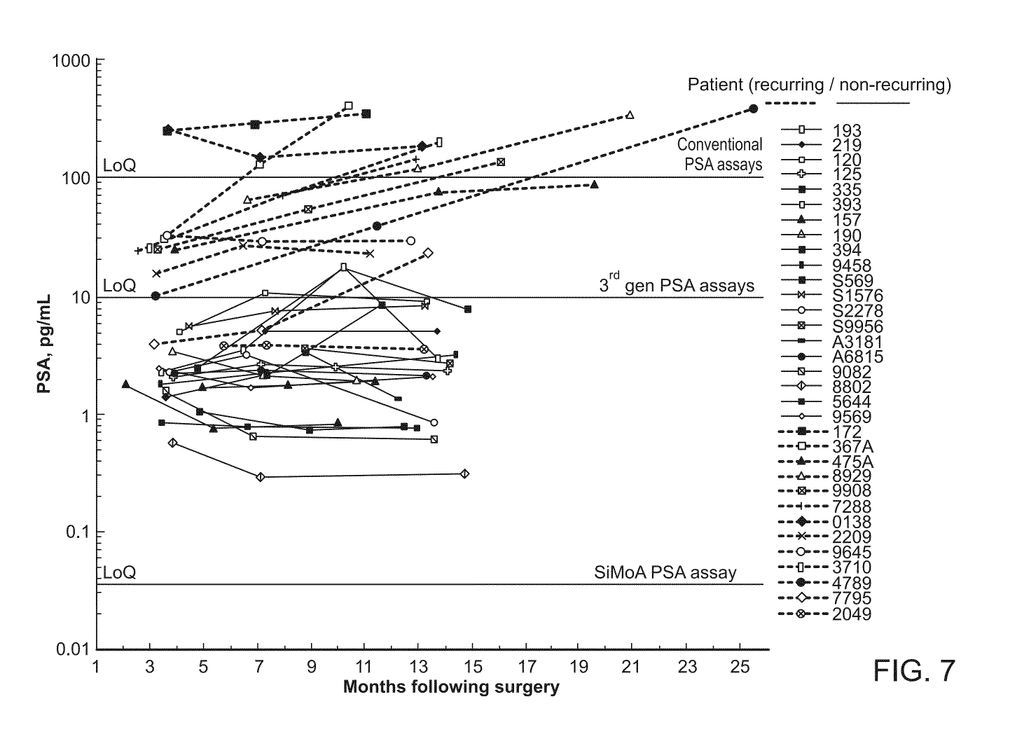

CLINICAL SAMPLES: PSA results from all samples are shown in FIG. 7. Approximately half of the initial PSA values were below the LOQ for commercially available third-generation assays (LOQ ˜10 pg/mL), but all samples were at least 10-fold above the LOQ of this assay. Replicate CVs were consistent with the 10-day precision study. FIG. 7 highlights the relationship between the nadir PSA and BCR: all patients with a nadir above 10 pg/mL experienced biochemical relapse (dashed lines), while all patients with a nadir below 1 pg/mL remained BCR-free for at least five years (solid lines). Bifurcation of the data with a cut point between 1 and 10 pg/mL provided 100% sensitivity for predicting five year BCR-free survival (e.g., see Example 3). The optimal cut-off point is below the measurement capability of ultrasensitive PSA assays, therefore improvement in clinical sensitivity may be possible from reliable PSA quantification in the formally “undetectable” category. Slopes of PSA increase were also calculated as the median pairwise slope for each patient. Using a multivariate Cox proportional hazards model comprehending demographic, clinicopathologic, and PSA covariates, PSA nadir was a significant predictor of BCR-free survival (p<0.01), while PSA slope was not a significant predictor (p>0.05).

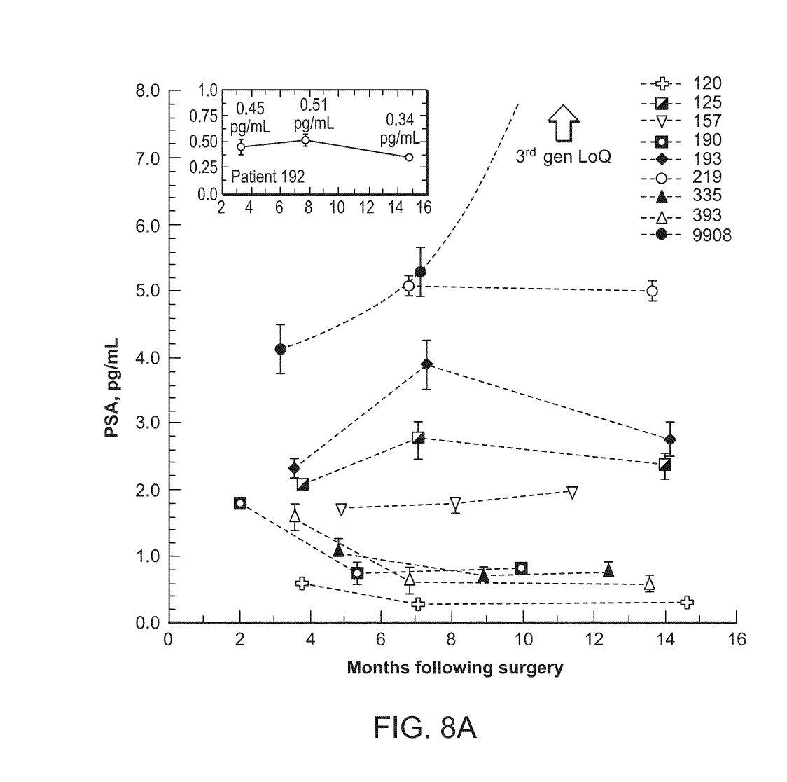

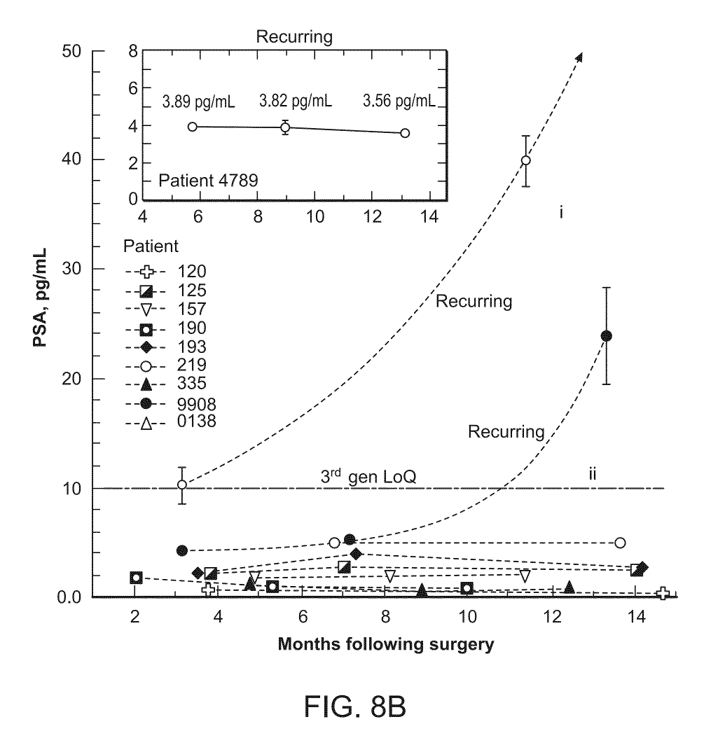

FIG. 8a highlights longitudinal data from five year BCR-free survivors from one of the clinical sites. All patients exhibited extremely low, stable PSA levels over the first year following surgery. Biological noise was minimal; for example, PSA values for patient 192 were 0.45, 0.51, and 0.34 pg/mL, a difference of only 0.17 pg/mL across 12 months (FIG. 8a inset). In contrast, there were other examples of non-recurrent patients (patients S9956, 9082, FIG. 7) exhibiting transient elevations to over 10 pg/mL, followed by PSA reduction back toward the nadir level. A similar phenomenon of lessor magnitude was noted in patients 193 and 125 (FIG. 8a). In contrast, patient 9908 (FIG. 8a, solid circles) exhibited a rapid upturn in PSA toward BCR from a similarly ultra low PSA level. Since these patients exhibited similar pre-surgical clinicopathologies (Tlc, Gleason Score 5-6, negative margins), factors contributing to successful remission in one patient in contrast to another at these ultra low PSA levels may include surgical, biological, and immunological variables.

FIG. 8b contrasts the non-recurring patients of FIG. 8a with three examples of recurring patients. While patient 9908 exhibited pre-surgical clinicopathology consistent with many non-recurrers, patient 0138 exhibited less favorable pathology (T2b, pT3b with seminal vescicle invasion, Gleason 7). The more aggressive recurrers (see FIG. 7) tended to have less favorable pre-surgical pathologies, particularly as regards pathological stage.

Patient 4789 exhibited highly stable, very low PSA values for 13 months following surgery (FIG. 8b inset), yet was diagnosed with BCR five years later. This patient had organ-confined disease (T2a, pT2c), with a Gleason Score of 9. Unpredictable remission and kinetic characteristics may complicate use of PSA velocity following RP for prediction of long-term recurrence.

Discussion: The data presented here indicate that the assay described in this example can define a new analytical standard for extremely sensitive and reproducible PSA testing. Historically, acceptance of ultrasensitive PSA measurement has been inhibited by analytical variability, which has reduced the reliability of the information obtained from these assays. Monitoring PSA after RP is analytically demanding because it requires both sensitivity and day-to-day reproducibility. Compounding the difficulty has been confusion over “analytical sensitivity” (LOD) and true quantification sensitivity (LOQ). Assessing day-to-day reproducibility of results from ultra-low test samples is the most rigorous means of understanding an assay's quantification sensitivity. This example demonstrates analytically acceptable day-to-day reproducibility in the sub-picogram range, low enough for reliable quantification of PS A in RP patients.

Robust fifth-generation measurement of PSA in all RP patients has the potential to impact management of prostate cancer in a number of significant ways. Reports showing the prognostic value of nadir PSA suggest a category of patients may be identified that represent an extremely low likelihood for BCR. Data from the study reported here indicate that a subgroup of patients below the detection limit of current methods were recurrence-free after five years. As reflected in FIG. 8, PSA levels appear biochemically stable for non-recurrent men. Current practices of looking for undetectable PSA levels using less sensitive detection methods could be supplanted with reliable data indicating a highly favorable status. Positively discerning these patients with precise measurement of their PSA levels could improve delineation of an ultra low risk category with statistically powered follow-up studies.

PSA trends measured with increased sensitivity could provide the earliest possible indicator of potential aggressive BCR, with significant potential improvement in early warning time relative to current PSA methods. As shown in FIG. 8b, an exponential rise in PSA would not have been measured by a third-generation assay in patient 9908 for 11 months following surgery. Salvage radiation therapy (SRT) is more effective if administered earlier rather than later in the cancer recurrence. Generally, intervention at the earliest sign of recurrence is most likely to lead to the most favorable outcome.

Reliably measuring PSA in every RP patient with fifth-generation sensitivity could also provide additional guidance on who may benefit most from adjuvant radiation treatment (ART). Evidence is growing of significant increases in overall and cancer-specific survival after ART. However, only about a third of patients who have had RP develop BCR, and about a third of this subset develop metastases, Which patients would benefit from ART and which patients would be over-treated remains unclear. Lower risk pathology with nadir PSA in an ultra low risk group might represent a cohort for whom ART represents over treatment. Higher risk pathology with high nadir could be a group most likely to benefit from ART. Treatment decisions for patients between these two groups could be better informed by highly reliable post surgical PSA data.

FIGURES

FIG. 2. Dose-response and linearity of the PSA assay.

Y-axis refers to the average number of label enzymes per individual microbead captured in the array. Fitting for optimal read-back utilized four-parameter logistical regression. FIG. 3a highlights the low background obtained with digital quantification. 20 calibration curves gave a mean signal:background ratio at 0.1 pg/mL of 4.33. FIG. 3b depicts linearity obtained from admixtures of high and low female serum samples.

FIG. 4. Limit of Quantification (LOQ) of the PSA assay.

LOQ was defined as the concentration of PSA at which measurement variation over time reached 20%. LOQ was estimated by non-linear power fit of sample replicate CVs across six weeks of testing. The equation of the fit gave a LOQ of 0.0352 pg/mL (standard error 0.0340-0.0387 pg/mL). Female serum samples are shown in grey circles.

FIG. 5. Reproducibility of the PSA assay.

Total imprecision was estimated by repeated measurement of a panel of prepared PSA samples over a 10-day period with two runs/day. Variation sources included fiber strips and processing, inter-calibration, and day-to-day reproducibility. The lowest sample was prepared to approximate the LOQ, and the total imprecision obtained was consistent with the LOQ estimate (20% CV at 0.035 pg/mL).

FIG. 6. Accuracy of the PSA assay.

Accuracy was assessed by comparison to a commercially available equimolar PSA method standardized with WHO reference material. Linear regression statistics across the 48 serum samples were=1.01(Centaur)+0.0025, R²=0.970 (standard error of the estimate 0.53). The sample set included RP patients drawn soon after surgery prior to PSA clearance, leading to elevated values. FIG. 6a highlights inter-method agreement extending to the lowest levels measurable by the Centaur. FIG. 6b shows absence of significant bias (mean bias 0.024 pg/mL),

FIG. 7. Post RP PSA results.

Longitudinal samples were tested from 13 recurring (dashed lines) and 18 non-recurring (solid lines) RP patients. All samples were well above the LOQ of the assay and were measured with good precision. Horizontal lines depict LOQs. The initial PSA value (nadir PSA) was a significant predictor of 5-year biochemical recurrence-free survival (p<0.01), while PSA slope was not a significant predictor in this study (p>0.05).

FIG. 8. Select longitudinal PSA trends.

FIG. 8a depicts PSA results from non-recurring patients from one of the clinical sites. Most patients exhibited extremely low, stable PSA levels over the first year following surgery. The early stages of BCR for patient 9908 (solid circles) are also depicted. The LOQ of ultrasenstive PSA methods is off the scale (arrow). FIG. 8b compares the same non-recurring patients with three examples of recurring patients (lines i and ii) on a broader scale. Exponential projections for the appearance of 200 pg/mL PSA for patients 9908 and 0138 (curved fits, R² 0.999) were consistent with actual BCR. Inset depicts PSA results from a patient in remission who later recurred.

### EXAMPLE 3

INTRODUCTION: Prostate specific antigen (PSA) is a serine protease produced almost exclusively by the epithelial elements of the prostate. Serum assays detecting PSA were first approved by the FDA in 1986 for monitoring prostate cancer after treatment. It wasn't until 1994 that an assay measuring serum PSA was approved by the FDA for the early detection of prostate cancer in combination with digital-rectal examination (DRE). A major limitation of the use of PSA for screening/early detection is its lack of specificity due to its production by benign as well as malignant prostatic epithelium.

Theoretically, PSA should be a useful tool for monitoring the effectiveness of radical prostatectomy (RP) to eradicate the disease since all of the prostate tissue should be removed. Residual local spread or systemic metastases of prostate cancer following RP manifests as a measurable PSA level which increases over time depending on the extent of disease. A recent study provides compelling evidence that benign residual prostate tissue is a very rare cause of measureable PSA after RP.

There are several potential advantages of predicting residual disease following RP based on the nadir or initial post-prostatectomy PSA measurement. First, those men who are destined not to develop disease recurrence may be reassured soon after RP, thereby alleviating anxiety and the need for extended monitoring. Second, those destined to develop recurrent disease may be offered adjuvant treatment if clinically indicated at an earlier time point.

The assay methods used in this example have a limit of PSA quantification <0.01 pg/mL, which is 1000 fold lower than conventional ultrasensitive PSA assays. The primary objective of this proof of concept study was to determine the utility of the nadir post prostatectomy levels for predicting 5 year biochemical free survival following RP.

Methods

Patients: A total of 31 frozen serum specimens were obtained from specimen logs of men who had undergone open radical retropubic prostatectomy (ORRP) with a minimum of 5 years PSA follow up for those without evidence of biochemical recurrence. For all men, a serum specimen was obtained between 3 and 6 months following ORRP which are referred to as the nadir sample. All specimens were required to have had a PSA level of <0.1 ng/mL measured by conventional PSA methods at the time of serum collection for entry into the study. Patients with evidence of nodal or distant metastases at the time of surgery were excluded from the study. No subjects received neo-adjuvant or adjuvant hormonal or radiation treatment.

Baseline demographic information, preoperative serum PSA, clinical stage, Gleason score of the prostate biopsy, pathologic stage and Gleason score, surgical margin status, PSA nadir and subsequent PSA levels, date of BCR , and date of any secondary prostate cancer treatment was maintained prospectively as part of longitudinal prospective IRB approved databases and specimen biorepositories at the respective institutions. Biochemical recurrence was defined as two consecutive PSA>0.2 ng/ml after the initial collected sample or secondary treatment for progressively rising serum PSA.

All serum samples were kept frozen at -70° C. or colder from the time of initial collection. Specimens were shipped on dry ice for testing. Assay methods: The assay method employed is a single molecule digital enzyme-linked immunosorbent assay with sub-femtomolar detection limits of serum PSA. Briefly described, this technology detects single protein molecules in blood by capturing the proteins on microscopic beads decorated with specific antibodies and labeling the immunocomplexes with a reporter capable of generating a fluorescent product (e.g., see Example 2). After isolating the beads in 50-femtoliter reaction chambers designed to hold a single bead, fluorescence imaging detects the single protein molecules. The assay method has been shown to provide linear response over approximately four logs of concentration ([PSA] from 8 fg/mL to 100 pg/mL) and extends a dynamic range from picomolar levels down to subfemtomolar levels in a single measurement. For description of various details associated with this assay, see, Example 4. Additional information may be found, for example, in U.S. Patent Application No. US 2011-0212848 (Ser. No. 12/731,130), filed Mar. 24, 2010, entitled “ULTRA-SENSITIVE DETECTION OF MOLECULES OR PARTICLES USING BEADS OR OTHER CAPTURE OBJECTS,” by Duffy et al.; International Patent Publication No. WO2011/109364 (International Patent Application No. PCT/US2011/026645), filed Mar. 1, 2011, entitled “ULTRA-SENSITIVE DETECTION OF MOLECULES OR PARTICLES USING BEADS OR OTHER CAPTURE OBJECTS,” by Duffy et al.; International Patent Publication No. WO2011/109372 (International Patent Application No. PCT/US2011/026657), filed Mar. 1, 2011, entitled “ULTRA-SENSITIVE DETECTION OF MOLECULES USING DUAL DETECTION METHODS,” by Duffy et al.; U.S. Patent Application No. US 2011-0212462 (Ser. No. 12/731,135), filed Mar. 24, 2010, entitled “ULTRA-SENSITIVE DETECTION OF MOLECULES USING DUAL DETECTION METHODS,” by Duffy et al.; International Patent Publication No. WO2011/109379 (International Patent Application No. PCT/US2011/026665), filed Mar. 1, 2011, entitled “METHODS AND SYSTEMS FOR EXTENDING DYNAMIC RANGE IN ASSAYS FOR THE DETECTION OF MOLECULES OR PARTICLES,” by Rissin et al.; U.S. Patent Application No. US 2011-0212537 (Ser. No. 12/731,136), filed Mar. 24, 2010, entitled “METHODS AND SYSTEMS FOR EXTENDING DYNAMIC RANGE IN ASSAYS FOR THE DETECTION OF MOLECULES OR PARTICLES,” by Duffy et al.; U.S. Patent Application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.; U.S. Patent Application No. US 2011-0245097 (Ser. No. 13/037,987), filed Mar. 1, 2011, entitled “METHODS AND SYSTEMS FOR EXTENDING DYNAMIC RANGE IN ASSAYS FOR THE DETECTION OF MOLECULES OR PARTICLES,” by Rissin et al.; each herein incorporated by reference.

Statistical methods: A Cox proportional hazard model was performed to determine whether PSA level predicted risk of biochemical recurrence. The covariates that were entered into the regression equation were age at radical prostatectomy, pre-surgical PSA value (ng/ml), biopsy Gleason Score, clinical Stage (T1.T2), nadir value of the assay method (pg/ml), pathological Gleason Score, pathological stage (pT2, pT3) and margin status (negative, positive). Forward elimination using the likelihood ratio test was employed. Significance was set at p<0.05.

A bootstrapped 95% confidence interval for the nadir PSA value in the non-recurrence group was used to determine the PSA cut-off value that defined two risk groups.

Kaplan-Meier survival curves, stratified by the bifurcated PSA nadir value were used to examine time to 5 year biochemical recurrence after radical prostatectomy. A Student t-test was employed to determine the difference between mean nadir PSA values for those patients who recurred within 5 years and those patients who did not. All analyses were performed using SPSS, Version 18 (IBM, NY).

RESULTS: The recorded characteristics of the 31 men undergoing ORRP fulfilling the study criteria are summarized in Table 1. Overall, 11 (35.5%) developed a BCR. The relevant characteristics are compared between the recurrence and non-recurrence groups. Age at RP and race were similar amongst the groups. The group of men who developed a BCR, recurred within a mean of 2.1 years from RP and had higher pre-surgical PSA, clinical and pathological stage, Gleason score and grade than the group of non-recurrent men. Margin status was similar amongst the groups.

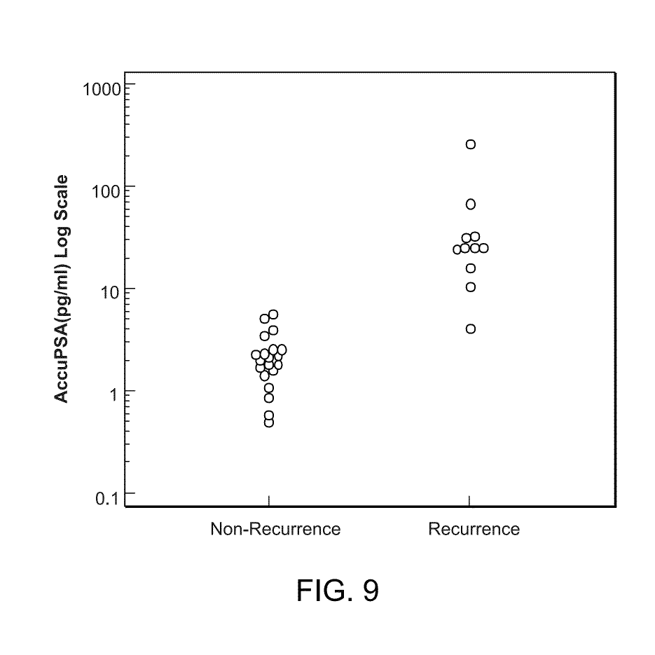

The distribution of the nadir PSA levels and the nadir PSA statistics for the recurrence and non recurrence groups are shown in FIG. 9 and Table 2, respectively. The mean PSA level in the non-recurrence and recurrence groups were 2.27 pg/mL and 46.99 pg/mL, respectively (p<0.001). Although PSA<0.1 ng/mL was a study inclusion criteria, nadir values for two patients exceeded this value as measured by this assay methods and were not excluded from the study. Differences in standardization and high variability at the detection limit of the conventional PSA methods could account for this discrepancy.

Cox-multivariate regression analysis was performed and nadir PSA was an independent predictor of BCR (Table 3). The parameter estimate for this PSA assay (B=0.014) indicates that at any given time the risk of recurring will increase by 1.01% for every one pg/ml increase in the nadir PSA level.

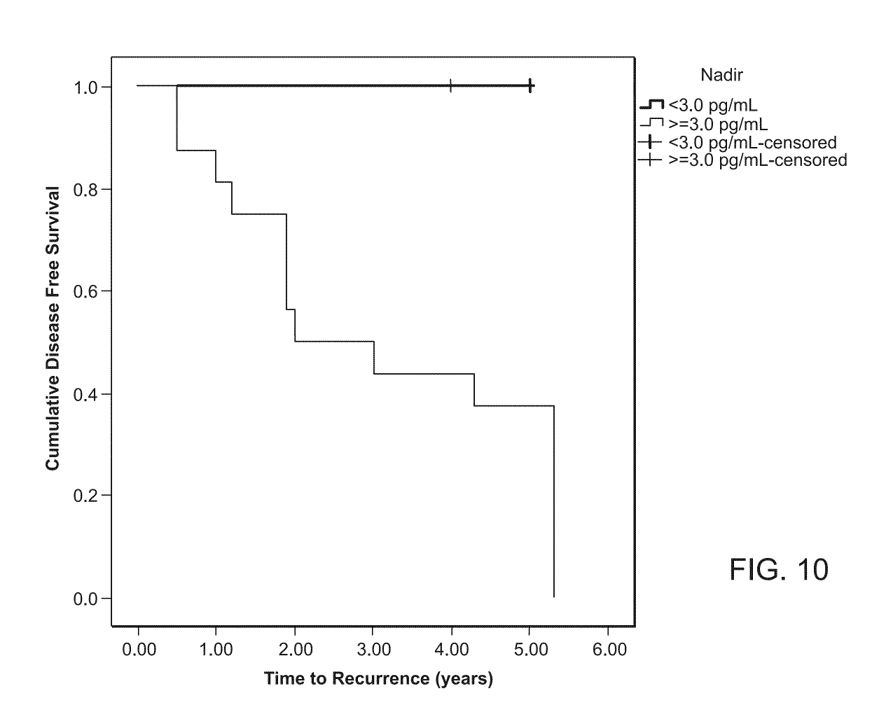

A bootstrapped 95% confidence interval for the nadir PSA level has an upper limit of 2.9 pg/ml. The value of 3.0 pg/ml was used as a cut point to define two risk groups (high vs low) for BCR. The Kaplan Meier survival curves for the risk groups defined by the bifurcated nadir value is shown in FIG. 10. The p-value for the difference in BCR free survival between the two plots is 0.00024.

The derivation of the sensitivity, specificity, positive predictive value (PPV), and negative predictive value (NPV) for a nadir PSA cut-point of 3 pg/ml for predicting BCR within 5 years is shown in Table 4. The sensitivity, specificity, PPV and NPV was 100%, 75%, 69% and 100%, respectively.

DISCUSSION: This study was designed to investigate whether a single nadir ultrasensitive PSA would predict BCR following RP. The nadir PSA level was significantly higher in the group who developed BCR. This assay methods and analysis was an independent predictor of BCR. The cut-point of 3.0 pg/ml provided the best separation between the recurrence and non-recurrence groups.

There are two scenarios where this PSA assay method and analysis has the opportunity to impact the post-prostatectomy management. First, is the ability to reassure an individual that he has truly a low risk of disease recurrence which is reflected in the negative predictive value. In the present study, the negative predictive value of a PSA level <3 pg/ml was 100%. The other opportunity of this assay method and analysis was to identify men earlier for adjuvant treatment which is reflected in the specificity of the assay. In the present study the specificity of assay methods was about 75%.

The major strength of the present study is that all men had appropriately stored frozen serum for a nadir PSA determination. The 5 years follow up time interval may be sufficient to identify most cases of clinically significant BCR.

TABLE 1
Characteristics of men undergoing RP stratified
by Recurrent and Non-Recurrent groups

| | | Non- | | | |
|---|---|---|---|---|---|
| | | Recurrence | | Recurrence | |
| | | (n = 20) | | (n = 11) | |
| Age at RP (mean years) | | 61.5 | | 61.0 | |
| Time to event (mean years) | | 5.0 | | 2.1 | |
| Pre-surgical PSA (mean ng/ml) | | 6.4 | | 10.3 | |
| Race (n, %) | Caucasian | 19 | (95) | 10 | (91) |
| | African | | | 1 | (9) |
| | American | | | | |
| | Unknown | 1 | (5) | | |
| Gleason Score (n, % ) | 5 | 1 | (5) | | |
| | 6 | 13 | (65) | 4 | (36) |
| | 7 | 3 | (15) | 7 | (64) |
| | 8 | 3 | (15) | | |
| Clinical Stage (n, %) | T1c | 15 | (75) | 5 | (45) |
| | T2a | 4 | (20) | 3 | (27) |
| | T2b | 1 | (5) | 3 | (27) |
| Gleason Grade (n, %) | Missing | | | 1 | (9) |
| | 2 + 3 | 1 | (5) | | |
| | 3 + 3 | 13 | (65) | 4 | (36) |
| | 3 + 4 | 1 | (5) | 3 | (27) |
| | 4 + 3 | 2 | (10) | 3 | (27) |
| | 4 + 4 | 3 | (15) | | |
| Pathology Stage (n, %) | pT2a | 4 | (20) | 1 | (9) |
| | pT2b | 6 | (30) | | |
| | pT2c | 7 | (35) | 1 | (9) |
| | pT3a | 1 | (5) | 5 | (45) |
| | pT3b | | | 3 | (27) |
| | Missing | 2 | (10) | 1 | (9) |
| Margin Status (n, %) | Negative | 19 | (95) | 10 | (91) |
| | Positive | 1 | (5) | 1 | (9) |
| Pathology Gleason Grade | Missing | 1 | (5) | | |
| (n, %) | 3 + 2 | 1 | (5) | 1 | (9) |
| | 3 + 3 | 10 | (50) | 1 | (9) |
| | 3 + 4 | 5 | (25) | 5 | (45) |
| | 4 + 3 | | | 2 | (18) |
| | 4 + 4 | 1 | (5) | | |
| | 4 + 5 | 1 | (5) | 2 | (18) |
| | 5 + 4 | 1 | (5) | | |

TABLE 2
Nadir PSA levels stratified by Recurrent and Non-recurrent groups

| | | Non- | |
|---|---|---|---|
| | | Recurrence | Recurrence |
| | | (pg/mL) | (pg/mL) |
| N | | 20 | 11 |
| Mean | | 2.27 | 46.99 |
| Std. Deviation | | 1.36 | 71.50 |
| Range | | 5.14 | 253.03 |
| Minimum | | 0.49 | 4.11 |
| Maximum | | 5.63 | 257.14 |
| Percentiles | 25th | 1.46 | 15.87 |
| | 50th | 2.04 | 25.04 |
| | 75th | 2.52 | 32.47 |

TABLE 3
Cox Regression Analysis of Biochemical Recurrence
Variables in the Equation

| | | B | SE | Wald | df | Sig. | Exp(B) |
|---|---|---|---|---|---|---|---|
| Step 1 | Nadir PSA | .019 | .007 | 7.842 | 1 | .005 | 1.019 |
| Step 2 | Pathological | 2.025 | .841 | 5.801 | 1 | .016 | 7.579 |
| | Stage | | | | | | |
| | (pT2, pT3) | | | | | | |
| | Nadir PSA | .014 | .007 | 4.417 | 1 | .036 | 1.014 |

TABLE 4
Sensitivity, Specificity, Positive predictive value (PPV), and Negative
predictive value (NPV) of [PSA] at a cutoff of 3 pg/mL

| | Recurrence | ≧5 yr. | | |
|---|---|---|---|---|
| | within 5 yr. | Non-Recurrence | | |
| [PSA] ≧3 pg/mL | 11 | 5 | 69% | PPV |
| [PSA] <3 pg/mL | 0 | 15 | 100% | NPV |
| | 100% | 75% | | |
| | Sensitivity | Specificity | | |

### EXAMPLE 4

This example provides additional details regarding the assay methods employed in Examples 1, 2, and 3.

Preparation of femtoliter-volume well arrays. Optical fiber bundles (Schott North America) approximately 5 cm long were sequentially polished on a polishing machine (Allied High Tech Products) using 30-, 9-, and 1-μm -sized diamond lapping films. The polished fiber bundles were chemically etched in a 0.025 M HCl solution for 130 s, and then immediately submerged into water to quench the reaction. The etched fibers were sonicated for 5 s in water, washed in water for 5 min, and dried under vacuum. The differential etch rate of the core and cladding glass of the fiber bundle arrays caused 4.5-μm-diameter wells to be formed in the core fibers. Different etch times resulting in different well depths were initially investigated. If wells were too deep, then multiple beads were deposited in each well and sealing was disrupted; if wells were too shallow, then the beads were not retained in the wells and poor loading efficiencies were observed. Well depths of 3.25+/−0.5 μm were optimal for retaining single beads in wells while maintaining good seals.

Reagents. Three reagents were developed: paramagnetic Aβ42 capture beads, biotinylated detector, and a streptavidin:β-galactosidase (SβG) conjugate. The capture beads were comprised of a monoclonal anti-PSA antibody (BiosPacific) directed to amino acid residues 158-163. The antibody was covalently attached by standard coupling chemistry to 2.7 μm carboxy paramagnetic microbeads (Varian). Individual beads are captured in array wells 4.5 μm wide×3.25 μm deep. It was important that the capture beads remain monomeric. The antibody-coated beads were diluted to a working concentration of 5×10⁶ beads/ml in Tris buffer with a surfactant and BSA. Biotinylated detector reagent was comprised of a monoclonal anti PSA antibody (BiosPacific) directed to amino acid residues 3-11. The antibody was biotinylated using standard methods and diluted to a concentration of 0.15 m/ml in a PBS diluent containing a surfactant and newborn calf serum, NCS (PBS/NCS). SβG was prepared by covalent conjugation of purified streptavidin (Thermo Scientific) and βG (Sigma) using standard coupling chemistry. For assay, aliquots of a concentrated βG stock were diluted to 15 pM in PBS/NCS with 1 mM MgCl2.Aliquots of a concentrated stock solution of SβG were prepared in PBS with 50% glycerol and were stored at −20° C. Prior to assay, an aliquot was thawed and diluted to 25 pM in PBS/NCS with 1 mM MgCl2.

Assay. Bead-sample incubations and labeling of immunocomplexes in conical 96 well plates (Axygen) were conducted using a robotic liquid handling system (Tecan EVO 150). Conical wells were used to facilitate magnetic attraction of the beads to the sides of the wells for efficient removal of reaction mixtures and bead washing. For magnetic attraction, a microplate bar magnet (Invitrogen) was used. Incubation periods were conducted with shaking on a microplate shaker (VWR) to keep beads suspended in the wells. The assays were initiated by mixing 100 μL of sample with 500,000 capture beads, and the mixtures were incubated with shaking for two hours. Plasma samples were pre-diluted 1:4 prior to assay with PBS/BSA with a heterophilic blocking agent as a precaution for sample quality and interference effects. Following incubation, the beads were washed three times with a wash buffer of 5-fold concentrated PBS with a surfactant (5×PBS). Biotinylated detector antibody (100 μL) was then added and incubated with the beads for 45 minutes. After a second sequence of three washes with 5× PBS, 100 μL of SβG was incubated for 30 minutes to form the enzyme-labeled immunocomplex. The beads were then washed six times per above, and concentrated to 2×10⁷ beads/mL with the addition of a reduced volume (25 μ×L) of array loading buffer comprised of PBS with a surfactant.

Loading of beads into femtoliter-volume well arrays. A short length of PVC tubing was placed on the etched end of a fiber bundle to create a reservoir to hold the bead solution. Ten microliters of the concentrated bead solution from the ELISA assay were pipetted into this reservoir. The fiber bundle was then centrifuged at 1,300 g for 10 min to force the beads into the etched wells. The PVC tubing was removed after centrifugation. The fiber bundle was dipped in PBS solution to wash off excess bead solution, and the surface was swabbed with deionized water. In addition to well depth (see above), bead concentration was an important parameter for maximizing bead loading efficiencies. Above concentrations of 200,000 beads per 10 μL loaded, typically 40-60% of wells in a 50,000-well array were occupied by a single bead, resulting in percentage active beads with acceptable Poisson noise. At concentrations below 200,000 beads per 10 μL loaded, bead loading efficiency dropped, resulting in fewer active beads and higher Poisson noise. In these experiments, at least 200,000 beads per reaction were used and loaded onto the arrays.

Detection of beads and enzyme-labeled beads in femtoliter-volume well arrays. A custom-built imaging system containing a mercury light source, filter cubes, objectives, and a CCD camera was used for acquiring fluorescence images. Fiber bundles were mounted on the microscope stage using a custom fixture. A droplet of β-galactosidase substrate (RGP) was placed on the silicone gasket material and placed in contact with the well arrays. A precision mechanical platform moved the silicone sheet into contact with the end of the fiber bundle, creating an array of isolated femtoliter-volume reaction vessels. Fluorescence images were acquired (558 nm excitation; 577 nm emission) with an exposure time of 1011 ms. Five frames (at 30-s intervals) were taken for each femtoliter-volume well array. The product of the enzymatic reaction used in these studies—resorufin—has high photostability with a low photobleaching rate (rate of photobleaching, kph=0.0013 s-1)21, making multiple exposures possible. Time-course fluorescence measurements were performed (i) to allow stable fluorescent artifacts to be removed from images, and (ii) to ensure that the signal from a beaded well was from an enzyme. For (i), the first fluorescent image was subtracted from fluorescent images acquired at each subsequent time point. This process removed light intensity that did not change with time, for example, fluorescence from dust and scattered light. For (ii), a positive or “on” well was identified only where fluorescence intensity in a beaded well increased in every frame, and by at least 20% over four frames. This process removed false positives from random changes in fluorescence during image acquisition.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements.

This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of determining a patient's prognosis for recurrence of prostate cancer and/or determining a course of treatment for prostate cancer, following a radical prostatectomy, comprising:
performing an assay on a sample obtained from the patient following the radical prostatectomy to determine a measure of the concentration of prostate specific antigen (PSA) in the sample, wherein the concentration of PSA in the sample is less than about 50 pg/mL; and
determining the patient's prognosis for recurrence of prostate cancer and/or determining a course of treatment for prostate cancer following the radical prostatectomy based at least in part on the measured concentration of PSA in the sample, wherein determining the patient's prognosis for recurrence of prostate cancer and/or determining a course of treatment does not require measurement of a change in concentration of PSA measured in multiple patient samples as a function of time elapsed after the radical prostatectomy.

2-3. (canceled)

4. A method of determining a patient's prognosis for recurrence of prostate cancer, and/or determining a course of treatment for prostate cancer following a radical prostatectomy, comprising:
performing an assay on a sample obtained from the patient following the radical prostatectomy to determine a measure of the concentration of prostate specific antigen (PSA) in the sample, wherein the sample is obtained from the patient within 6 months following the radical prostatectomy; and
determining the patient's prognosis for recurrence of prostate cancer and/or determining a course of treatment for prostate cancer following a radical prostatectomy based at least in part on the concentration of PSA measured in the sample, wherein determining the patient's prognosis for recurrence of prostate cancer and/or determining a course of treatment does not require measurement of a change in concentration of PSA measured in multiple patient samples as a function of time elapsed after the radical prostatectomy.

5-6. (canceled)

7. A method of determining a patient's prognosis for recurrence of prostate cancer, and/or determining a course of treatment for prostate cancer following a radical prostatectomy, comprising:
performing an assay on at least one sample obtained from the patient following the radical prostatectomy to determine a measure of the concentration of prostate specific antigen (PSA) in the at least one sample; and
determining the patient's prognosis for recurrence of prostate cancer and/or determining a course of treatment for prostate cancer following a radical prostatectomy based at least in part on the concentration of PSA measured in the at least one sample, wherein a measured concentration of PSA in the at least one sample greater than a threshold limit of no greater than about 10 pg/mL indicates a significant likelihood that the patient's prostate cancer will reoccur within 5 years.

8-12. (canceled)

13. The method of claim 1, wherein the at least one sample is a bodily fluid.

14. The method of claim 13, wherein the bodily fluid is blood or a blood component.

15. The method of claim 13, wherein the blood component is plasma or serum.

16. The method of claim 1, wherein 2, samples are obtained from the patient.

17. The method of claim 1, wherein the limit of quantification of the assay is less than about 9 pg/mL.

18. (canceled)

19. The method of claim 1, wherein measured concentration of PSA in the sample is less than about 40 pg/mL.

20. The method of claim, wherein the sample is obtained from the patient at or less than about 2 years following the radical prostatectomy.

21. The method of claim 4, wherein measured concentration of PSA in the sample is less than about 100 pg/mL.

22. The method of claim 4, wherein the sample is obtained from the patient within 5 months following the radical prostatectomy.

23. The method of claim 7, wherein the threshold limit is no greater than about 9 pg/mL.

24. (canceled)

25. The method of claim 7, wherein the at least one following a radical prostatectomy.

26. The method of claim 7, wherein a measured concentration of PSA greater than a threshold limit of no greater than about 10 pg/mL indicates that the patient's chance of recurrence of prostate cancer is at greater than about 50% within 5 years.

27. (canceled)

28. The method of claim 1, wherein the measured concentration of PSA is the patient's nadir PSA.

29. The method of claim 1, wherein the assay comprises:
spatially segregating at least a portion of the PSA molecules into a plurality of separate locations;
addressing at least a portion of the plurality of locations subjected to the spatially segregating step and determining the number of said locations containing a PSA molecules; and
determining a measure of the concentration of PSA in the sample based at least in part on the number of locations determined to contain a PSA molecules.

30. The method of claim 29, further comprising exposing the plurality of PSA molecules to a plurality of binding ligands such that at least some of the PSA molecules associate with a single binding ligand and a statistically significant fraction of the PSA molecules do not associate with any binding ligand;

31. The method of claim 1, wherein the assay comprises:
immobilizing PSA molecules with respect to a plurality of capture objects such that at least some of the capture objects associate with at least one PSA molecule and a statistically significant fraction of the capture objects do not associate with any PSA molecules;
spatially segregating at least a portion of the capture objects subjected to the immobilizing step into a plurality of separate locations;
addressing at least a portion of the plurality of locations subjected to the spatially segregating step and determining the number of said locations containing a PSA molecule; and
determining a measure of the concentration of PSA in the sample based at least in part on the number of locations determined to contain a PSA molecule.

32. The method of claim 1, wherein the assay comprises:
exposing a plurality of capture objects that each include a binding surface having affinity for PSA, to a solution containing or suspected of containing PSA;
immobilizing PSA molecules with respect to the plurality of capture objects such that at least some of the capture objects associate with at least one PSA molecule and a statistically significant fraction of the capture objects do not associate with any PSA molecules;
spatially segregating at least a portion of the capture objects subjected to the immobilizing step into a plurality of separate locations;
addressing at least a portion of the plurality of locations subjected to the spatially segregating step and determining the number of said locations containing a PSA molecule; and
determining a measure of the concentration of PSA in the sample based at least in part on the number of locations determined to contain a PSA molecule.

33-49. (canceled)
